(19) United States
(12) Patent Application Publication
(10) Pub. No.: US 2024/0076697 A1
(43) Pub. Date: Mar. 7, 2024

(54) METHODS FOR INCREASED NUCLEIC ACID-GUIDED CELL EDITING

(71) Applicant: Inscripta, Inc., Pleasanton, CA (US)

(72) Inventors: Karl GERHARDT, Westminster, CO (US); Charles JOHNSON, Arvada, CO (US); Martha BRAUN, Westminster, CO (US)

(73) Assignee: Inscripta, Inc., Pleasanton, CA (US)

(21) Appl. No.: 18/240,550

(22) Filed: Aug. 31, 2023

Related U.S. Application Data
(60) Provisional application No. 63/403,235, filed on Sep. 1, 2022.

(51) Int. Cl. C12N 15/90; C12N 1/20; C12N 1/38; C12N 9/22; C12N 15/11; C12N 15/70

(52) U.S. Cl. CPC C12N 15/902; C12N 1/20; C12N 1/38; C12N 9/22; C12N 15/11; C12N 15/70; C12N 2310/20; C12N 2800/101; C12N 2800/80; C12N 2830/002

(57) ABSTRACT

The present disclosure provides compositions of matter, methods, systems, and instruments for improved nucleic acid-guided nuclease editing in live cells, wherein the live cells are shifted into a growth-arrested state for editing.

### CROSS REFERENCED TO RELATED APPLICATIONS

This application claims the benefit of U.S. Provisional Application No. 63/403,235, filed Sep. 1, 2022, which is incorporated by reference herein in its entirety.

### FIELD

The present disclosure relates to compositions of matter, methods, systems, and instruments for nucleic acid-guided editing in live cells, and more particularly, to nucleic acid-guided editing in live cells that have reached a growth-arrested state.

### BACKGROUND

In the following discussion, certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequence, and hence, aerie function. These nucleases include nucleic acid-guided nucleases and nuclease fusions, which enable researchers to generate permanent edits in live cells. Generally, it is desirable to attain the highest editing rates possible in a cell population. However, many current methods have low editing efficiencies, which is partially attributed to the high toxicity of editing-induced double-stranded breaks in target genomic DNA. This toxicity facilitates the enrichment of non-edited cells, or editing “escapees,” leading to low editing rates in resultant cell populations.

Accordingly, there is a need in the art for improved methods, compositions, systems, and instruments for nucleic acid-guided nuclease editing. The present disclosure addresses this need.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In certain embodiments, a method for performing nucleic acid-guided nuclease editing in a genome of a live cell is provided, the method comprising: providing an editing system to a cell with a target locus, the editing system comprising: (a) (i) a nucleic acid-guided nuclease or (ii) a vector encoding the nucleic acid-guided nuclease; (b) (i) a gRNA recognizing the target locus or (ii) a nucleic acid encoding the gRNA; and (c) a donor template comprising a desired edit to the target locus; inducing the cell into a growth-arrested state; and providing conditions to allow the editing system to introduce the desired edit into the target locus.

In certain embodiments, a method for performing nucleic acid-guided nuclease editing in a genome of a live cell is provided, the method comprising: providing a cell with a target locus; transforming the cell with an editing system, the editing system comprising:

(a) (i) a nucleic acid-guided nuclease or (ii) a vector encoding the nucleic acid-guided nuclease; (b) (i) a gRNA recognizing the target locus or (ii) a nucleic acid encoding the gRNA; and (c) a donor template comprising a desired edit to the target locus; inducing the cell into a growth-arrested state; providing conditions to allow the editing system to introduce the desired edit into the target locus; and inducing the cell into a growth state.

These aspects and other features and advantages of the present disclosure are described below in more detail.

### BRIEF DESCRIPTION OF THE DRAWINGS

The foregoing and other features and advantages of the present disclosure will be more fully understood from the following detailed description of illustrative embodiments taken in conjunction with the accompanying drawings in which:

FIG. 1A is a simplified block diagram of an exemplary method for improved editing of live cells wherein the cells are shifted into a growth-arrested state for editing, according to embodiments of the present disclosure.

FIG. 1B illustrates an exemplary method for inducing and recovering a live cells into/from a growth-arrested state for editing, according to embodiments of the present disclosure. FIG. 1C illustrates an exemplary growth curve for cells in culture (optical density versus time) during the method of FIG. 1B, according to embodiments of the present disclosure.

FIGS. 2A-2C depict three different views of an exemplary automated multi-module cell processing instrument for performing trackable nucleic acid-guided nuclease editing employing a split protein reporter system.

FIG. 3A depicts one embodiment of a rotating growth vial for use with the cell growth module described herein and in relation to FIGS. 3B-3D. FIG. 3B illustrates a perspective view of one embodiment of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (bottom) members for use in a tangential flow filtration module (e.g., cell growth and/or concentration module), as well as the retentate and permeate members assembled into a tangential flow assembly (bottom). FIG. 4B depicts two side perspective views of a reservoir assembly of a tangential flow filtration module. FIGS. 4C-4E depict an exemplary top, with fluidic and pneumatic ports and gasket suitable for the reservoir assemblies shown in FIG. 4B.

FIG. 5A depicts an exemplary combination reagent cartridge and electroporation device (e.g., transformation module) that may be used in a multi-module cell processing instrument. FIG. 5B is a top perspective view of one embodiment of an exemplary flow-through electroporation device that may be part of a reagent cartridge. FIG. 5C depicts a bottom perspective view of one embodiment of an exemplary flow-through electroporation device that may be part of a reagent cartridge. FIGS. 5D-5F depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing and normalizing cells in a solid wall device. FIGS. 6B-6D depict an embodiment of a solid wall isolation incubation and normalization (SWIIN) module. FIG. 6E depicts the embodiment of the SWIIN module in FIGS. 6B-6D further comprising a heater and a heated cover.

FIG. 7 is a simplified process diagram of an embodiment of an exemplary automated multi-module cell processing instrument comprising a solid wall singulation/growth/editing/normalization module for recursive and trackable cell editing—including mammalian cell editing.

FIG. 8 illustrates process conditions during editing of six different editing runs, wherein cells in 5/6 of the editing runs were induced into a growth-arrested state.

FIGS. 9A-9C illustrate the number of recovered colony-forming units (CFUs), the fraction of inert cells (i.e., non-edits), and the edit rate, respectively, for each of the six editing runs depicted in FIG. 8.

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W.H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this disclosure belongs. All publications mentioned herein are incorporated by reference for the purpose of describing and disclosing devices, methods and cell populations that may be used in connection with the presently described disclosure.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

In the following description, numerous specific details are set forth to provide a more thorough understanding of the present disclosure. However, it will be apparent to one of ordinary skill in the art that the present disclosure may be practiced without one or more of these specific details. In other instances, well-known features and procedures well known to those skilled in the art have not been described in order to avoid obscuring the disclosure.

The term “growth-arrested state” as used herein refers to a state of halted progression of a cell through the cell cycle, wherein cellular processes such as genome duplication and/or cell division are stopped. Such states may be chemically, physically, or genetically induced by exogenous or endogenous stimuli.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

The terms “CREATE fusion editing” or “CF editing” in the context of the current methods and compositions refers to an editing technique that uses a nuclease editing enzyme having nickase activity in conjunction with one or more nucleic acids to facilitate editing. In specific embodiments, CF editing methods utilize a fusion protein, such as a nucleic acid-guided nickase/reverse transcriptase fusion, and a nucleic acid encoding one or more editing gRNAs comprising a region complementary to a target region of a nucleic acid. The one or more gRNAs are covalently linked to a repair template comprising a region homologous to the target region and having a mutation, e.g., an edit, of at least one nucleotide. For information regarding CF editing see, e.g., U.S. Pat. Nos. 10,689,669; 11,268,078; 11,268,088; U.S. Ser. No. 16/740,421; and PCT Nos. PCT/US2020/023725 and PCT/US2019/048607.

The terms “CREATE fusion editing cassette” or “CF editing cassette” in the context of the current methods and compositions refers to a nucleic acid molecule comprising a coding sequence for transcription of a CREATE fusion gRNA or “CFgRNA” to effect editing in a nucleic acid-guided nickase/reverse transcriptase fusion system where the CFgRNA is designed to bind to and facilitate editing of one or both DNA strands in a target locus.

The terms “CREATE fusion editing components” or “CF editing components” refers to one or both of a nucleic acid-guided nickase enzyme/reverse transcriptase fusion protein (“nickase-RT fusion”) and a CREATE fusion editing cassette (“CF editing cassette”) and/or CREATE fusion gRNA (“CFgRNA”) to effect editing in live cells.

The terms “CREATE fusion gRNA” or “CFgRNA” refer to a single RNA molecule comprising two portions, the first portion being a gRNA and the second portion being a repair template covalently linked to the gRNA and comprising an edit to a target locus of a cell genome.

The terms “donor DNA” or “donor nucleic acid” or “donor polynucleotide” or “donor template” refer to a nucleic acid molecule designed to introduce an edit (e.g., insertion, deletion, substitution or other sequence modification) into a target locus by homology-directed repair, e.g., homologous recombination, using nucleic acid-guided nucleases and editing gRNAs. For homology-directed repair, the donor template must have sufficient homology to regions flanking the “cut site,” or site to be edited, in a genomic target sequence. A donor template generally comprises one or more homology arms to facilitate homology-directed repair. The length of the homology arm(s) will depend on, e.g., the type and size of the modification being made. In many instances, the donor DNA will have two regions of sequence homology (e.g., two homology arms) to the genomic target locus. Preferably, an “edit” region or “sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites.

The term “editing cassette” refers to a nucleic acid molecule comprising (i) a coding sequence for a guide nucleic acid or gRNA and (ii) a covalently linked donor template sequence.

The terms “guide nucleic acid” or “guide RNA” or “gRNA” refer to a polynucleotide comprising 1) a guide sequence (e.g., a “spacer” sequence”) capable of hybridizing to a target genomic locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on a donor DNA with a certain degree of homology with a target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The term “subminimal media” refers to mediums for maintaining cell populations that contain less than the minimum nutrients necessary for colony growth. Accordingly, a subminimal medium may be utilized to induce cells into a growth-arrested state. Generally, the number of ingredients that are added to a subminimal medium varies depending on which type of cell is being maintained/grown.

The term “nucleic acid” or “polynucleotide” refers to deoxyribonucleic acids (DNA) or ribonucleic acids (RNA) and polymers thereof in either single- or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with DNA, RNA, cDNA, gene, and mRNA encoded by a gene.

“Nucleic acid-guided editing components” refers to one, some, or all of a nuclease, a guide nucleic acid, a donor nucleic acid, and recombination systems, if required.

As used herein, “nucleic acid-guided nickase/reverse transcriptase fusion” or “nickase-RT fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase is fused to a reverse transcriptase, which is an enzyme used to generate cDNA from an RNA template. In certain embodiments, “nucleic acid-guided nickase/reverse transcriptase fusion” or “nickase-RT fusion” refers to two or more nucleic acid-guided nickases—or nucleic acid-guided nucleases or CRISPR nucleases that have been engineered to act as nickases rather than nucleases that initiate double-stranded DNA breaks—where the nucleic acid-guided nickases are fused to a reverse transcriptase. For information regarding nickase-RT fusions see, e.g., U.S. Pat. No. 10,689,669 and U.S. Ser. No. 16/740,421.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA.

As used herein, the terms “protein,” “peptide,” and “polypeptide” are used interchangeably herein and refer to a polymer of amino acid residues. Proteins may or may not be made up entirely of amino acids transcribed by any class of any RNA polymerase I, II or III. Promoters may be constitutive or inducible.

As used herein, the term “repair template” in the context of a CREATE fusion editing system employing a nickase-RT fusion enzyme refers to a nucleic acid that is designed to serve as a template (including a desired edit) to be incorporated into target DNA via reverse transcriptase.

As used herein the term “selectable marker” refers to a gene introduced into a cell, which confers a trait suitable for artificial selection. General use selectable markers are well known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonoacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target genomic DNA locus”, “target locus”, or “target genomic locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The term “variant” may refer to a polypeptide or polynucleotide that differs from a reference polypeptide or polynucleotide (e.g., a wild-type) but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In the present disclosure, a single vector may include a coding sequence for a nickase-RT fusion enzyme and a CF editing cassette and/or CFgRNA sequence to be transcribed. In other embodiments, however, two vectors—e.g., an engine vector comprising the coding sequence for the nickase-RT fusion enzyme, and an editing vector, comprising the CFgRNA sequence to be transcribed—may be used.

The present disclosure relates to compositions of matter, methods, systems, and instruments for improved nucleic acid-guided editing in live cells. More particularly, with the present compositions and methods, improved editing rates are facilitated via editing of live cells that have reached a growth-arrested state, wherein the cells are non-growing (e.g., non-replicating) but are still able to respond to external signals, activate transcription, and produce proteins. Accordingly, because the cells are growth-arrested, the toxicity of editing-induced double stranded breaks and other editing-related events may be reduced, as the cells have more time to “recover” from such events since they may not be actively growing and/or dividing.

Thus, in some aspects, there is provided a method for performing nucleic acid-guided nuclease editing in a genome of a live cell, the method comprising: providing an editing system to a cell with a target locus, the editing system comprising: (a) (i) a nucleic acid-guided nuclease or (ii) a vector encoding the nucleic acid-guided nuclease; (b) (i) a gRNA recognizing the target locus or (ii) a nucleic acid encoding the gRNA; and (c) a donor template comprising a desired edit to the target locus; inducing the cell into a growth-arrested state; and providing conditions to allow the editing system to introduce the desired edit into the target locus.

In some aspects, there is provided a method for performing nucleic acid-guided nuclease editing in a genome of a live cell, the method comprising: providing a cell with a target locus; transforming the cell with an editing system, the editing system comprising:

(a) (i) a nucleic acid-guided nuclease or (ii) a vector encoding the nucleic acid-guided nuclease; (b) (i) a gRNA recognizing the target locus or (ii) a nucleic acid encoding the gRNA; and (c) a donor template comprising a desired edit to the target locus; inducing the cell into a growth-arrested state; providing conditions to allow the editing system to introduce the desired edit into the target locus; and inducing the cell into a growth state.

In some aspects, the growth-arrested state of the cells is induced via exposure of the cells to one or more stress conditions (or “stresses”), including chemical and/or physical stresses. Examples of suitable stress conditions include, but are not limited to nutrient limitation, chemical/drug exposure, pH change, temperature change, high concentration(s) of organic acid(s), osmotic stress, oxidative stress, and the like, which may be provided individually or in combination.

In some aspects, the growth-arrested state of the cells is induced via starvation of the cells.

In specific aspects, the growth-arrested state of the cells is induced via exposure and/or incubation of the cells in subminimal media. In specific aspects, a subminimal medium comprises one or more salts, glucose, amino acids, and water. In such aspects, the one or more salts may comprise one or more of magnesium sulfate (MgSO₄), calcium dichloride (CaCl₂)), ammonium chloride (NH₄Cl), sodium chloride (NaCl), monopotassium phosphate (KH₂PO₄), and sodium phosphate dibasic heptahydrate (Na₂HPO₄). In specific aspects, the subminimal medium comprises M9-type subminimal medium (e.g., M9 medium+/−desired ingredients). Generally, M9 medium comprises one or more of M9 salts, glucose, casamino acids, MgSO₄, and CaCl₂). In specific aspects, the subminimal medium comprises Davis-type Broth (e.g., Davis Minimal Broth+/−desired ingredients).

In some aspects, the donor template comprises a donor polynucleotide, such as a donor DNA template, which may be separate from the gRNA. In some aspects, a donor DNA template may comprise one or more regions of complimentary (e.g., homology arms) to a sequence of the target locus for incorporation of the desired edit via homology-directed repair, such as homologous recombination.

In some aspects, the gRNA is a component of an editing cassette for performing nucleic acid-guided nuclease editing, the editing cassette comprising a sequence encoding the gRNA and a sequence encoding the donor template with the desired edit for incorporation into the target locus of the cell genome. In some aspects, the components of the editing cassette are contiguous. In some aspects, the editing cassette is agnostic to the order of the sequence encoding the gRNA and the sequence encoding the donor template. In some aspects, the gRNA is under the control of a promoter of the editing cassette.

In some aspects, the donor template or the editing cassette further comprises an edit (e.g., 1, 2, 3, 4, 5, or up to 10 edits) to immunize the target locus to prevent re-nicking or re-cutting thereof by the nucleic acid-guided nuclease. As discussed herein, in some aspects, an edit to immunize a target locus to prevent re-cutting is one that alters the proto-spacer adjacent motif (or other element) such that subsequent binding at the target locus by the nucleic acid-guided nuclease is impaired or prevented. In some aspects, the donor template or the editing cassette further comprises an amplification priming site or subpool primer binding sequence at, e.g., a 3′ end thereof. In specific aspects, the donor template or the editing cassette further comprises a melting temperature booster sequence at, e.g., a 5′ end thereof, which is a short protective DNA buffer sequence. In addition, in specific aspects, the donor template or the editing cassette comprises regions of homology to a vector for gap-repair insertion of the donor template or the editing cassette into the vector, such as an editing vector or engine vector.

In some aspects, a region of complementarity between the gRNA and the target locus is from 4-120 nucleotides in length, or from 5-80 nucleotides in length, or from 6-60 nucleotides in length, e.g., from 0-10 nucleotides in length, 10-20 nucleotides in length, 20-50 nucleotides in length, or 50-100 nucleotides in length.

In some aspects, the edit is from 1-750 nucleotides in length, or from 1-500 nucleotides in length, or from 1-150 nucleotides in length, e.g., from 1-10 nucleotides in length, 10-20 nucleotides in length, 20-50 nucleotides in length, 50-100 nucleotides, 100-250 nucleotides, 250-500 nucleotides, or 500-750 nucleotides in length.

In some aspects, the donor template or the editing cassette comprises two or more edits, or three or more edits, or four or more edits, or five or more edits.

In some aspects, an edit includes one or more base swaps in the target locus.

In some aspects, an edit includes an insertion in the target locus.

In some aspects, an edit includes an insertion of recombinase sites, protein degron tags, promoters, terminators, alternative-splice sites, CpG islands, etc.

In some aspects, an edit created includes a deletion in the target locus.

In some aspects, an edit is designed to provide a deletion of from 1 to 750 nucleotides at the target locus. In some aspects, an edit is designed to provide a deletion of from 1 to 10 nucleotides, from 10 to 20 nucleotides, from 20 to 50 nucleotides, from 50 to 100 nucleotides, from 100 to 200 nucleotides, from 200 to 500 nucleotides or from 250 to 750 nucleotides at the target locus.

In some aspects, an edit includes a deletion of introns, exons, repetitive elements, promoters, terminators, insulators, CpG islands, non-coding elements, retrotransposons, etc.

In some aspects, an edit comprises several types of edits and/or comprises more than one of one or more types of edits. For example, in some aspects, an edit comprises two or more base swaps (e.g., 2, 3, 4, 5, or from 1 to 20 base swaps), some or all of which can be adjacent to each other or nonadjacent to each other. In some aspects, an edit comprises one or more base swaps (e.g., 2, 3, 4, 5, or from 1 to 20 base swaps) and an insertion of one or more nucleotides (e.g., 2, 3, 4, 5, or from 1 to 20 nucleotides). In some aspects, an edit comprises one or more base swaps (e.g., 2, 3, 4, 5, or from 1 to 20 base swaps) and a deletion of one or more nucleotides (e.g., 2, 3, 4, 5, or from 1 to 20 nucleotides).

In some aspects, an edit is directed to a coding region in the target locus.

In some aspects, an edit is directed to a noncoding region in the target locus.

In some aspects, the donor template or the editing cassette further comprises one or more barcodes or other unique molecular identifiers (UMIs) to facilitate tracking of edits via sequencing. In such aspects, each different edit may correspond with a different barcode or UMI.

In some aspects, the nucleic acid-guided nuclease includes a MAD-series nuclease, nickase, or a variant (e.g., orthologue) thereof. In some aspects, the nuclease includes a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nuclease, nickase, variants thereof, and/or combinations thereof.

In some aspects, the nucleic acid-guided nuclease includes a Cas9 nuclease (also known as Csn1 and Csx12), nickase, or a variant thereof.

In some aspects, the nuclease includes C2c1, C2c2, C2c3, Cas1, Cas1B, Cas2, Cas3, Cas4, Cas5, Cash, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, or similar nuclease, nickase, variants thereof, and/or combinations thereof.

In some aspects, the gRNA (or editing cassette) and the nuclease are introduced into the cell on a single vector (e.g., a single-part system). In certain embodiments, the gRNA and/or the nuclease are introduced into the cell as a multi-part system, wherein the gRNA may be introduced separately from the nuclease. For example, the gRNA may be comprised on a first vector, and the nuclease may be comprised on a second vector co-delivered with the first vector.

In some aspects, the nuclease is introduced into the cells as a DNA molecule coding for the nuclease separately or linked to the gRNA, or the nuclease may introduced separately in protein form or as part of a complex. In some aspects, the gRNA and the nuclease are introduced into the cells as a ribonucleoprotein (RNP) complex.

In some aspects, the gRNA and/or the nucleic acid-guided nuclease are introduced into the cell on a linear or circular plasmid. In some aspects, the gRNA and/or the nucleic acid-guided nuclease are under the control of a constitutive or inducible promoter at a 5′ end thereof.

In some aspects, a vector comprising the gRNA and/or the nucleic acid-guided nuclease further comprises an origin of replication and a selectable marker component, e.g., an antibiotic resistance gene or a fluorescent protein gene, for selection or enrichment of cells that have been edited. The selectable marker may be utilized for selective enrichment of edited. In some aspects, the selectable marker comprises an antibiotic resistance gene or a fluorescent protein. In some aspects, the selectable marker comprises the PuroR gene.

In some aspects, there is provided a library of vector or plasmid backbones, and/or a library of gRNAs (e.g., in editing cassettes), to be transformed into cells. In some aspects, the utilization of a library of cassettes and/or a library of vector or plasmid backbones enables combinatorial or multiplex editing in the cells. A library of cassettes or vectors may comprise cassettes or vectors that have any combination of common elements and non-common or different elements as compared to other cassettes or vectors within the pool. For example, a library of editing cassettes can comprise common priming sites or common homology regions, while also containing non-common or unique edits. Combinations of common and non-common elements are advantageous for multiplexing or combinatorial techniques disclosed herein.

In some aspects, a library of editing cassettes comprises at least 2 cassettes, or at least 10 cassettes, or at least 100 cassettes, or at least 1,000 cassettes, or at least 10,000 cassettes, or at least 100,000 cassettes, or at least 1,000,000 cassettes. In some aspects, a library of cassettes comprises from 5 to a 1,000,000 cassettes, or from 100 to 500,000 cassettes, or from 1,000 to 100,000 cassettes, or from 1,000 to 10,000 cassettes, or from 10,000 to 50,000 cassettes.

In some aspects, one or more editing cassettes in a library of editing cassettes each comprise a different gRNA targeting a different target locus within the cell genome. In some aspects, one or more editing cassettes in a library of editing cassettes each comprise a different edit to be incorporated within the cell genome.

In some aspects, there is provided a library comprising a plurality of editing cassettes or a plurality or vectors comprising cassettes as disclosed herein. In some aspects, within the library are distinct editing cassette and barcode combinations, which when sequenced upon editing, facilitate tracking of editing events in a population of cells. Accordingly, when edits and barcodes are incorporated into a target genome, the incorporation of an edit is determined based on sequenced the barcode.

In some aspects, there is provided a gene-wide or genome-wide library of cassettes or vectors comprising a cassettes as disclosed herein.

In some aspects, there are provided methods of recursive or iterative rounds of editing operations. In some aspects, during each round of editing, a new or unique edit (and, in some embodiments, a corresponding barcode) is incorporated into the cell genome, such that multiple editing rounds may be used to construct combinatorial diversity throughout the genome. In such aspects, sequencing of barcodes can be used to reconstruct each combinatorial genotype or to confirm that the edit from each round or operation has bene incorporated into the genome. In some aspects, methods disclosed herein comprise 2 or more rounds of editing, such as 5 or more rounds of editing, such as 10 or more rounds of editing.

In some aspects, the cells are induced into a continuous growth-arrested state during which multiple recursive or iterative rounds of editing operations are performed. In some aspects, the cells are induced into a growth-arrested state during each round for performance of a recursive or iterative editing process.

In some aspects, one or more unique barcodes can be inserted in each round of multiple iterative or recursive editing operations. In such aspects, the unique barcodes may be inserted adjacent or in proximity to each other (e.g., in a single target region), or at a distance and/or in separate target regions.

In some aspects, recursive or iterative editing methods may be used for analyzing combinatorial mutational effects on large populations, or for inserting entire pathways within cells.

The present disclosure includes methods of nucleic acid-guided nuclease editing in cell populations, e.g., prokaryotic, archaeal, and eukaryotic cells. In some aspects, the cells include bacterial cells. In some aspects, the cells include fungal cells. In some aspects, the cells include mammalian cells.

In specific aspects, the cells include E. coli cells.

The present disclosure provides, in select embodiments, compositions and methods for use with modules, instruments, and systems of automated multi-module cell processing systems configured to perform nucleic acid-guided genome editing in multiple cells. Automated systems for cell processing that may be used with embodiments of the present disclosure can be found, e.g., in U.S. Pat. Nos. 10,253,316; 10,329,559; 10,323,242; 10,421,959; 10,465,185; 10,519,437; 10,584,333; 10,584,334; 10,647,982; 10,689,645; 10,738,301; and 10,738,663.

In some aspects, the automated multi-module cell processing instruments are designed for recursive genome editing, e.g., sequentially introducing multiple edits into genomes inside one or more cells of a cell population through two or more editing operations within the instruments.

### Nucleic Acid-Guided Nuclease Editing, Generally

Certain embodiments described herein utilize nucleic acid-guided nuclease editing (i.e., RNA-guided nuclease or CRISPR editing) for performing genomic editing in live cells, e.g., for performing the competitive editing assay described above, to screen for plasmid backbones. In some embodiments, one or more edits are introduced in a single round of editing utilizing a plurality, e.g., a library, of candidate plasmid backbones.

In CRISPR-type editing generally, a nucleic acid-guided nuclease or CREATE fusion enzyme complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

In general, a guide nucleic acid (e.g., a gRNA or CFgRNA) complexes with a compatible nucleic acid-guided nuclease or CREATE fusion enzyme and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In certain embodiments, the gRNA may be encoded by a DNA sequence on a plasmid, or the coding sequence may and preferably does reside within an editing cassette assembled into a plasmid backbone. Methods and compositions for designing and synthesizing editing cassettes and libraries of editing cassettes are described in U.S. Pat. Nos. 10,240,167; 10,266,849; 9,982,278; 10,351,877; 10,364,442; 10,435,715; 10,465,207; 10,669,559; 10,711,284; 10,731,180; and 11,078,498; all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably, the guide sequence is 10-30 or 15-20 nucleotides long, or 15, 16, 17, 18, 19, or 20 nucleotides in length.

In certain embodiments of the present methods and compositions, the guide nucleic acids are provided as mRNAs or as sequences to be expressed from a candidate plasmid (or vector), and/or as sequences to be expressed from a cassette optionally inserted into a plasmid backbone, and comprise both a guide sequence and a scaffold sequence as a single transcript under the control of a promoter, e.g., an inducible or constitutive promoter. In certain embodiments, the guide nucleic acid may be part of an editing cassette that encodes a repair template for effecting an edit in the cellular target sequence, and/or one or more homology arms. Alternatively, the guide nucleic acid may not be part of the editing cassette and instead may be encoded on the plasmid backbone. For example, a sequence coding for a guide nucleic acid can be assembled or inserted into a plasmid backbone first, followed by insertion of the repair template in, e.g., an editing cassette. In other cases, the repair template in, e.g., an editing cassette can be inserted or assembled into a plasmid backbone first, followed by insertion of the sequence coding for the guide nucleic acid. In certain embodiments, the sequence encoding the guide nucleic acid and repair template are located together in a rationally designed editing cassette and are simultaneously inserted or assembled via gap repair into a plasmid backbone to create an editing plasmid (i.e., an editing vector).

The guide nucleic acids are engineered to target a desired target sequence (e.g., a cellular “editing” target sequence) by altering the guide sequence so that the guide sequence is complementary to a desired target sequence, thereby allowing hybridization between the guide sequence and the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein), a non-coding sequence (e.g., a regulatory polynucleotide, an intron, a protospacer adjacent motif (PAM) sequence, or “junk” DNA), or other sequence.

In general, to generate an edit in the target sequence, a gRNA/nuclease complex binds to the target sequence as determined by the guide RNA, and the nuclease or CF enzyme recognizes a PAM sequence adjacent to or in proximity to the target sequence. The precise preferred PAM sequence and length requirements for different nucleic acid-guided nucleases vary; however, PAMs typically are 2-10 or so base-pairs in length and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease. In certain embodiments, genome editing of a cellular target sequence both introduces a desired DNA change to a cellular target sequence (an “intended” edit), e.g., the genomic DNA of a cell, and removes, mutates, or renders inactive a PAM region in the cellular target sequence (an “immunizing edit”), thereby rendering the target site immune to further nuclease binding. Rendering the PAM at the cellular target sequence inactive precludes additional editing of the cell genome at that cellular target sequence. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event may be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or CF enzyme component of the nucleic acid-guided nuclease or CF enzyme, a polynucleotide sequence encoding the nucleic acid-guided nuclease or CF enzyme can be codon optimized for expression in particular cell types, such as bacterial, yeast, and, here, mammalian cells. The choice of the nucleic acid-guided nuclease or CF enzyme to be employed depends on many factors, such as what type of edit is to be made in the target sequence and whether an appropriate PAM is located close to the desired target sequence. Nucleases of use in the methods described herein include but are not limited to Cas9, Cas12, MAD2, or MAD7, MAD2007 or other MADzymes and MADzyme systems (see U.S. Pat. Nos. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17,200,074; 17,200,089; 17/200,110; 16/953,233; 17/463,498; 63/134,938; 16/819,896; 17/179,193; and 16/421,783 for sequences and other details related to engineered and naturally-occurring MADzymes). CF enzymes typically comprise a CRISPR nucleic acid-guided nuclease engineered to cut one DNA strand in the target DNA rather than making a double-stranded cut, and the nickase portion is fused to a reverse transcriptase. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. A coding sequence for a desired nuclease or CF enzyme may be on an “engine vector” along with other desired sequences such as a selective marker(s), or a coding sequence for the desired nuclease or nickase may reside on an editing plasmid, or may be transfected into a cell as a protein.

Another component of the nucleic acid-guided nuclease system or CF system is the repair template comprising homology to the cellular target sequence. The repair template typically is designed to serve as a template for homologous recombination with a cellular target sequence cleaved by the nucleic acid-guided nuclease, or the repair template serves as the template for template-directed repair via the CF enzyme, as a part of the gRNA/nuclease complex. For the present methods and compositions, the repair template typically is on the same vector and, in certain embodiments, in the same editing cassette, as a guide nucleic acid for editing, and may be under the control of the same promoter as the gRNA (that is, a single promoter driving the transcription of both the gRNA and the repair template). A repair template polynucleotide may be of any suitable length, such as about or more than about 20, 25, 50, 75, 100, or more nucleotides in length. In certain preferred aspects, the repair template can be provided as an oligonucleotide of between 20-100 nucleotides, such as between 30-75 nucleotides. When optimally aligned, the repair template overlaps with (is complementary to) the cellular target sequence by, e.g., about 20, 25, 30, 35, 40, 50, 60, 70, 80, 90 or more nucleotides.

The repair template generally comprises two regions that are complementary to a portion of the cellular target sequence (e.g., homology arms). In certain embodiments of the present methods and compositions, the two homology arms flank an intended edit, e.g., at least one alteration as compared to the cellular target sequence, such as a DNA sequence insertion, which may be part of the repair template. In certain embodiments, the repair template comprises two homology arms that do not flank the intended edit. In such embodiments, the homology arms may be encoded on a plasmid backbone, or in an editing cassette with the edit.

Inducible editing is advantageous in that cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. It is this toxicity, however, that is exploited herein to perform curing.

An editing cassette may further comprise one or more primer binding sites. The primer binding sites are used to amplify the editing cassette by using oligonucleotide primers as described infra and may be biotinylated or otherwise labeled.

In addition, or alternatively to the edit, the editing cassette may comprise a barcode. A barcode is a unique DNA sequence that corresponds to the repair template such that the barcode can identify the edit made to the corresponding cellular target sequence. The barcode typically comprises four or more nucleotides.

In certain embodiments, the plasmid (and/or vector) encoding components of the nucleic acid-guided nuclease system, e.g., the candidate plasmid backbones, further encode a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

In certain embodiments, the plasmid backbones further comprise one or more selectable markers to enable artificial selection of cells undergoing editing and/or curing events. For example, in certain embodiments, the plasmid backbones encode for one or more antibiotic resistance genes, such as ampicillin/carbenicillin and chloramphenicol resistance genes, thereby facilitating enrichment for cells undergoing editing and/or curing events via depletion of the cell population. In other examples, plasmid backbones may include an integrated GFP gene to enable phenotypic detection of editing and/or curing events by flow cytometry, fluorescent cell imaging, etc.

In certain embodiments, engine and editing vectors may further comprise control sequences operably linked to the component sequences to be transcribed. As described above, promoters driving transcription of one or more components of the nucleic acid-guided nuclease editing system may be inducible. A number of gene regulation control systems have been developed for the controlled expression of genes in plant, microbe, and animal cells, including mammalian cells, such as the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In certain embodiments of the present methods used in the modules and instruments described herein, at least one of the nucleic acid-guided nuclease editing components (e.g., the nuclease and/or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media; to remove, e.g., an inducible biochemical in the medium that is used to induce editing.

Certain embodiments described herein may also utilize an alternative to traditional nucleic acid-guided nuclease editing (i.e., RNA-guided nuclease or CRISPR editing) for performing genome editing. For example, such embodiments may employ a nucleic acid-guided nickase/reverse transcriptase fusion enzyme (“nickase-RT fusion”) as opposed to a nucleic acid-guided nuclease (i.e., a “CRISPR nuclease”). The nickase-RT fusion differs from traditional CRISPR editing in that instead of initiating double-stranded breaks in the target genome and homologous recombination to effect an edit, the nickase initiates a nick in a single strand of the target genome, e.g., the non-complementary strand. Further, the fusion of the nickase to a reverse transcriptase, in combination with an editing cassette comprising a CFgRNA and repair template, e.g. a CF editing cassette, eliminates the need for a donor DNA to be incorporated by homologous recombination. Instead, the repair template of the corresponding cassette—typically a ribonucleic acid—may serve as a template for the reverse transcription (“RT”) portion of the fusion enzyme to add an intended to the nicked strand at the target locus. That is, utilization of a nickase-RT fusion enables incorporation of the edit in the target genome by copying an RNA sequence (i.e., at the RNA level) rather than replacing a portion of the target locus with a donor DNA (i.e., at the DNA level).

The nickase—functioning as a single-strand cutter and having the specificity of a nucleic acid-guided nuclease—engages the target locus and nicks a strand of the target locus creating one or more free 3′ terminal nucleotides. The 3′ end of the editing cassette is then annealed to the nicked strand, and the reverse transcriptase utilizes the 3′ terminal nucleotide(s) of the nicked strand to copy the repair template and create a “flap” containing the desired edit. Thereafter, endogenous repair mechanisms of the cells repair the nick in favor of the desired edit by hybridizing the flap to the wild-type (e.g., unedited) DNA strand.

### Improved Nucleic Acid-Guided Nuclease Editing of Growth-Arrested Cells

The present disclosure is drawn to increasing the efficiency of nucleic acid-guided nuclease editing by utilizing populations of cells in a growth-arrested state.

While the growing phases of cells, and particularly, bacterial cells, have been studied and characterized extensively, the non-growing phases thereof (e.g., stationary phase or quiescence) have been explored far less. Recently, however, it has been reported that bacterial cells can continue production of proteins at constant rates for several hours or even days upon entering into stationary phase, wherein the cells are non-growing, without significant decreases in cell viability. See Gefen et al., Proceedings of the National Academy of Sciences (PNAS), 111, 1 (2014). Accordingly, such findings indicate that at least bacterial cells, even when not growing, can maintain the constant ability to respond to external signals, activate transcription, and to produce proteins. See Gefen et al., PNAS, 111, 1 (2014).

The inventors of the present disclosure have discovered that the non-growing phases of cells, hereinafter referred to as “growth-arrested” states, can be leveraged to improve the outcomes of nucleic acid-guided nuclease editing. More particularly, by performing editing in growth-arrested cells, editing rates and efficiencies may be increased, as compared to editing cells in a growth phase or other phase of a corresponding cell cycle. It is hypothesized that because the cells are non-growing (e.g., non-replicating), the toxicity of editing-induced double stranded breaks and other editing-related events may be reduced, as the cells have more time to “recover” from such events, due to them not being actively growing and/or dividing. In other words, by pausing the “biological clock” of the cells, the cells have a longer window of opportunity to repair any toxic genomic effects of editing, rather than promptly entering a cell death pathway thereof. Additionally or alternatively, by performing editing in growth-arrested cells, editing cells in the population may deplete at lower rates relative to the inert/non-editing cells since both populations are not growing, thus allowing for the editing cells not to fall behind.

Accordingly, embodiments of the present disclosure provide compositions of matter, methods, systems, and instruments for improved nucleic acid-guided nuclease editing, wherein the edited cells comprised populations of growth-arrested cells. In certain embodiments, the growth-arrested states of the cells are induced, either during or prior to editing.

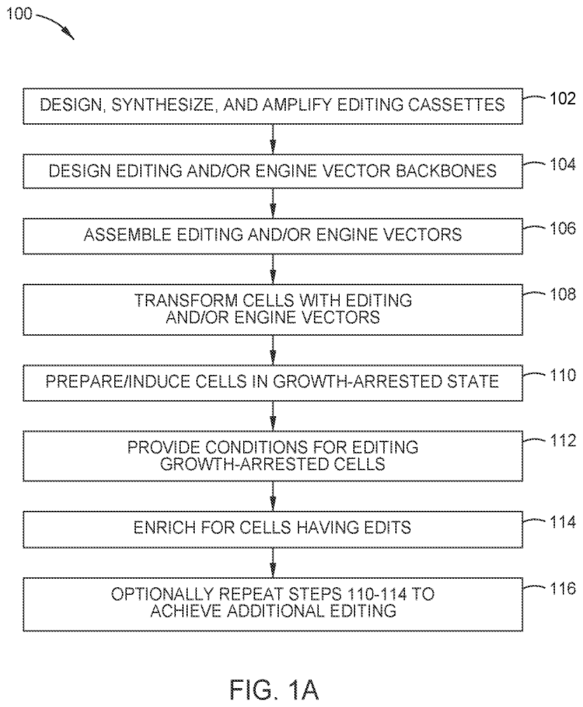

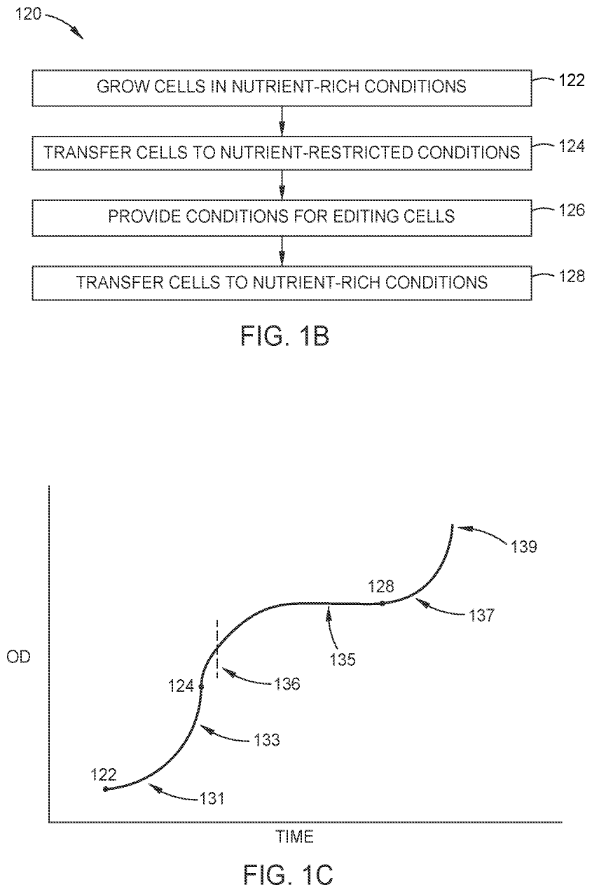

FIG. 1A is a simplified block diagram of an exemplary method 100 for nucleic acid-guided editing of live cells that have been induced into a growth-arrested state, according to embodiments of the present disclosure. Note that the exemplary method 100 is described with reference to nucleic acid-guided editing using a nucleic acid-guided nuclease in combination with an editing cassette comprising a gRNA and donor template; however, method 100 may also apply to other types of nucleic acid-guided editing methods, including nickase/reverse transcriptase fusion (“nickase-RT fusion”) editing, wherein a nickase-RT fusion enzyme is utilized in combination with a CREATE fusion editing cassette (“CF editing cassette”).

Looking at FIG. 1A, method 100 begins at 102 by designing and synthesizing editing cassettes, such as a library of editing cassettes, each comprising a covalently-linked editing gRNA and donor template designed to incorporate an edit into a target genomic locus of a cell population. In certain embodiments, each editing cassette further comprises a PAM and/or spacer mutation to render the PAM or spacer region of the target genomic locus inactive after editing. Once the editing cassettes have been synthesized, individual cassettes may be amplified. Again, methods and compositions for designing and synthesizing editing cassettes are described in U.S. Pat. Nos. 10,240,167; 10,266,849; 9,982,278; 10,351,877; 10,364,442; 10,435,715; and 10,465,207, all of which are incorporated herein in their entirety. U.S. Pat. No. 10,465,207 is drawn to multiplex or compound editing cassettes (e.g., two or more cassettes targeting different regions in the genome), which may be used with certain embodiments employed herein.

Similarly, at 104, which may be performed simultaneously with or subsequently to 102, editing and/or engine vector backbones are designed. In certain embodiments, the editing and/or engine vector backbones comprise plasmid backbones. Generally, an editing vector backbone typically comprises a selectable marker sequence, an origin of replication (e.g., a bacterial origin of replication), and/or other genetic elements. Meanwhile, an engine vector backbone comprises a coding sequence for a nucleic acid-guided nuclease (e.g., a coding sequence for MAD7), coding sequences for components of a recombineering system (e.g., the coding sequences for a λ Red recombineering system), an origin of replication, a selectable marker sequence (e.g., typically a different selectable marker than the selectable marker on the editing vector backbone), and/or other genetic elements. However, other vector backbone designs and arrangements are also contemplated. For example, in certain embodiments, a single vector backbone is designed for assembly with editing cassettes, the backbone comprising a coding sequence for a nucleic acid-guided nuclease, an origin of replication, a selectable marker, coding sequences for components of a recombineering system, and/or other genetic elements. In further embodiments, the single vector backbone may not comprise a nucleic acid-guided nuclease coding sequence, as the nuclease may instead be delivered to the cells as a protein or protein complex.

At 106, the editing cassettes are assembled with editing vector backbones to form editing vectors (e.g., a library of editing vectors), and the engine and/or editing vectors are thereafter combined and introduced into live cells at 108. Generally, the vectors are introduced into the cells as a single-vector or two-vector system. The cells at 108 may generally include any suitable cell type, including prokaryotic, archaeal, and eukaryotic cells. In certain embodiments, the cells include bacterial cells, such as E. coli cells. A variety of delivery systems may be used to introduce (e.g., transform, transfect, or transduce) nucleic acid-guided nuclease editing system components into a host cell 108. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in, e.g., U.S. Pat. No. 10,253,316, issued 9 Apr. 2019; U.S. Pat. No. 10,329,559, issued 25 Jun. 2019; U.S. Pat. No. 10,323,242, issued 18 Jun. 2019; U.S. Pat. No. 10,421,959, issued 24 Sep. 2019; U.S. Pat. No. 10,465,185, issued 5 Nov. 2019; U.S. Pat. No. 10,519,437, issued 31 Dec. 2019; U.S. Pat. No. 10,584,333, issued 10 Mar. 2020; U.S. Pat. No. 10,584,334, issued 10 Mar. 2020; U.S. Pat. No. 10,647,982, issued 12 May 2020; U.S. Pat. No. 10,689,645, issued 23 Jun. 2020; U.S. Pat. No. 10,738,301, issued 11 Aug. 2020; U.S. Pat. No. 10,738,663, issued 29 Sep. 2020; and U.S. Pat. No. 10,894,958, issued 19 Jan. 2021 all of which are herein incorporated by reference in their entirety.

At 110, the cells, now comprising the nucleic acid-guided nuclease editing system components, are induced into a growth-arrested state. In certain embodiments, the growth-arrested state of the cells is induced via exposure of the cells to one or more stress conditions (or “stresses”), including chemical and/or physical stresses. Examples of suitable stress conditions for induction include, but are not limited to: nutrient limitation (e.g., starvation), pH change, temperature change, high concentration(s) of organic acid(s), osmotic stress, oxidative stress, and the like. Generally, such stress conditions are non-lethal to the cells.

In certain embodiments, the growth-arrested state of the cells 110 is induced via transfer and/or incubation of the cells in subminimal medium which may “starve” the cells into the growth-arrested state. In certain embodiments, the cells may be washed into the subminimal media, or pelleted and then resuspended in the subminimal media to induce the growth-arrested state. As described above, a “subminimal medium” refers to any medium for maintaining cell populations that contain less than the minimum nutrients necessary for colony growth. Accordingly, the reduced nutrient content of such media may be utilized to induce cells into the growth-arrested state. In certain embodiments, a subminimal medium comprises or consists of one or more salts, glucose, amino acids, and water. In such embodiments, the one or more salts may comprise one or more of magnesium sulfate (MgSO₄), calcium dichloride (CaCl₂)), ammonium chloride (NH₄Cl), sodium chloride (NaCl), monopotassium phosphate (KH₂PO₄), and sodium phosphate dibasic heptahydrate (Na₂HPO₄). In specific embodiments, a subminimal medium comprises M9-type medium or Davis-type Broth.

In still other embodiments, the cells may be provided already in a growth-arrested state prior to transformation with the editing and/or engine vectors, or the cells may be induced into the growth-arrested state prior to transformation with the editing and/or engine vectors.

Once transformed 108 and in a growth-arrested state 110, conditions for nucleic acid-guided nuclease editing 112 of the growth-arrested cells are provided. “Providing conditions” includes incubation of the cells in appropriate medium and may also include providing conditions to induce transcription of an inducible promoter (e.g., adding antibiotics, adding inducers, increasing temperature) for transcription of an editing cassette and/or nucleic acid-guided nuclease.

Once editing is complete, the cells are allowed to recover and are preferably enriched for cells that have been edited 114. In certain embodiments, recovery includes removing the cells from the stress condition(s) that caused the cells to shift into the growth-arrested state, including nutrient limitation, pH, temperature, high concentrations or organic acids, osmotic stress, oxidative stress, etc. For example, in certain embodiments, the cells may be removed from nutrient-limited media and transferred onto nutrient-rich, non-minimal, or minimal media.

Enrichment can be performed directly, such as via cells from the population that express a selectable marker, or by using surrogates, e.g., cell surface handles co-introduced with one or more components of the editing machinery. At this point in method 100, the cells can be characterized phenotypically or genotypically (e.g., via sequencing) or, optionally, steps 102-114 or steps 110-114 may be repeated to make additional edits 116 in recursive or iterative editing rounds. In certain embodiments, steps 102-114 are repeated to create or construct a defined combination of edits or a combinatorial library.

After recovery and enrichment of edited cells, the genomic DNA or RNA transcripts of the cells may be sequenced to track or analyze the editing events, wherein any barcode(s) incorporated with the edits may serve as proxies for corresponding edits, or the edits themselves may be sequences. For example, the cells may be lysed and DNA or RNA extracted, purified, amplified, prepared into libraries, and sequenced to track for integrated barcodes and/or edits. In certain embodiments, amplicons of genomic DNA are sequenced via any suitable high-throughput method, such as single molecule real time (SNRT) sequencing, nanopore sequencing, sequencing by synthesis (SBS) or Illumina sequencing, Ion Torrent sequencing, sequencing by ligation (SBL), combinatorial probe anchor synthesis (cPAS) sequencing, parallel pyrosequencing, microfluidic methods, etc. In certain embodiments, the transcriptome of the cells is sequencing via any suitable high-throughput RNA sequencing (RNA-Seq) method, such as bulk or scRNA-Seq.

FIG. 1B illustrates an exemplary method 120 for inducing and recovering a population of cells into/from a growth-arrested state for editing (e.g., operations 108 and 116 of the method 100 described above), according to embodiments of the present disclosure. In the method 120, cells are shifted between growth and growth-arrested states using media having different levels of nutrients. Accordingly, method 120 exemplifies an embodiment of growth arrest of cells as induced by nutrient limitation. Generally, the method 120 may be utilized with any suitable types of cells, such as, e.g., bacterial cells, and the media (e.g., number/types of ingredients added to the media) and/or other nutrient conditions may be dependent on which type of cell is being edited.

As shown in FIG. 1B, method 120 beings at step 122, wherein cells are first grown in nutrient-rich conditions, such as on nutrient-rich media. These conditions promote an active growth state of the cells, such as log phase growth, wherein cells may be actively proliferating (e.g., dividing). Generally, the cells may be maintained in nutrient-rich conditions for any suitable period of time, such as 1, 2, 3, 4, 5, 6, 7, 8, 9, 10, or more hours, or until a suitable optical density (OD) of the cells is attained.

Thereafter, at step 124, the cells are transferred to nutrient-restricted conditions, such as onto subminimal media, to induce a growth-arrested state of the cells. Shifting into the growth-arrested state causes the cells to pause their growth, yet continue production of proteins. The onset of the growth-arrested state upon transfer of the cells to nutrient-restricted conditions may vary from cell type to cell type, based on the starvation dynamics of such cells.

Before, during, or after induction of the growth-arrested state, conditions for editing of the cells are provided 126, as described above with reference to method 100. Again, “providing conditions” may include providing conditions to induce transcription of an inducible promoter (e.g., adding antibiotics, adding inducers, increasing temperature) for transcription of an editing cassette and/or editing enzyme. In certain embodiments, such conditions may be provided after an initial period of cell exposure to nutrient-restricted conditions, such as an initial period of 0.5, 1, 1.5, 2, 2.5, 3, 3.5, 4, 4.5, 5, or more hours of the cells being exposed to nutrient-restricted conditions.

At 128, the cells are transferred back to nutrient-rich conditions, such as onto nutrient-rich media, for growth and recovery. Transfer of the cells back to conditions providing an abundance of nutrients promotes a shift of the cells back to the active growth state, wherein cells are actively proliferating. In certain embodiments, the nutrient-rich conditions at 128 may be the same nutrient-rich conditions at 122; in certain other embodiments, the nutrient-rich conditions at 128 are different than the nutrient-rich conditions at 122. In further embodiments, the nutrient-rich conditions at 128 may further comprise a selection/enrichment mechanism for cells that have been edited at 126. For example, where nutrient-rich media is used, the nutrient-rich media may also comprise one or more selection ingredients (e.g., antibiotics). Generally, the cells may be grown and recovered in the nutrient-rich conditions 128 for any suitable period of time, such as 1, 2, 3, 4, 5, 6, 7, 8, 9, 10, or more hours, or until a suitable optical density (OD) of the cells is attained for analysis and/or further editing.

FIG. 1C illustrates an exemplary growth curve 130 for cells in culture (optical density versus time) during the exemplary method 120, according to embodiments of the present disclosure. As shown, when the cells are grown in the nutrient-rich conditions at step 122, there is an initial lag phase 131, after which the cells enter log phase 133 where they grow quickly. Upon transfer to the nutrient-restricted conditions at 124, the cells shift into a growth-arrested state 135, where the cells are no longer dividing but are still constantly producing proteins. The present methods employ editing, e.g., step 126 in method 120 above, while the cells are in the growth-arrested state 135 or nearly so, such as at time point 136 or later in FIG. 1C. Thereafter, the cells are transferred back to nutrient-rich conditions at 128, wherein the cells enter another lag phase 137 and log phase 139 during growth and recovery.

### Automated Cell Editing Instruments and Modules to Perform Nucleic Acid-Guided Nuclease Editing in Cells

### Automated Cell Editing Instruments

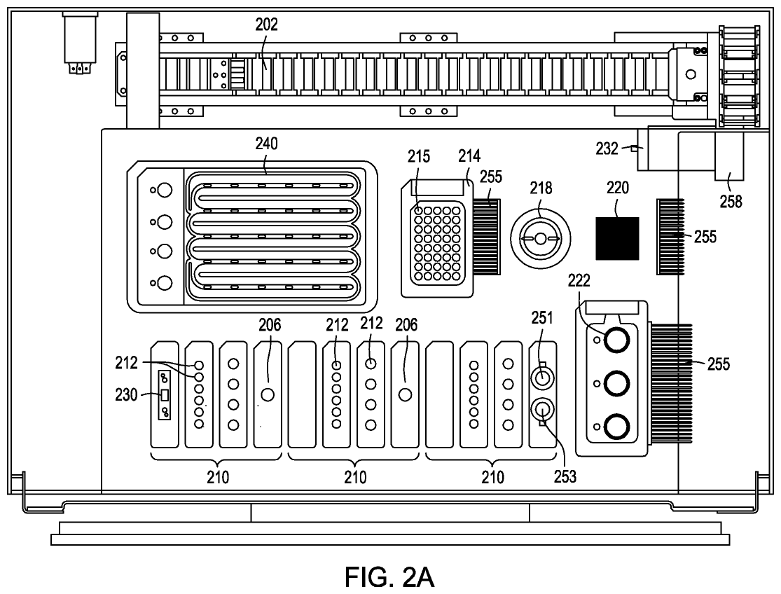

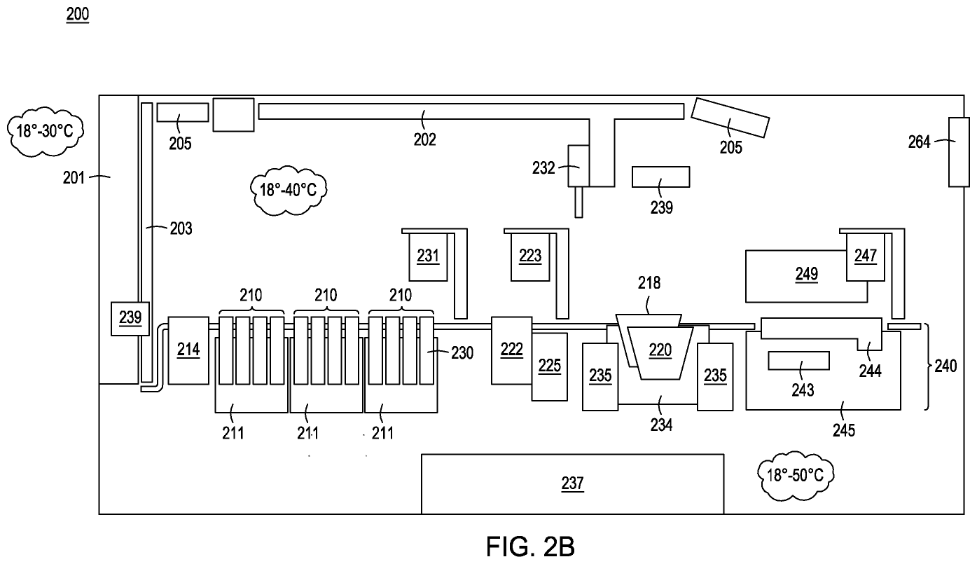

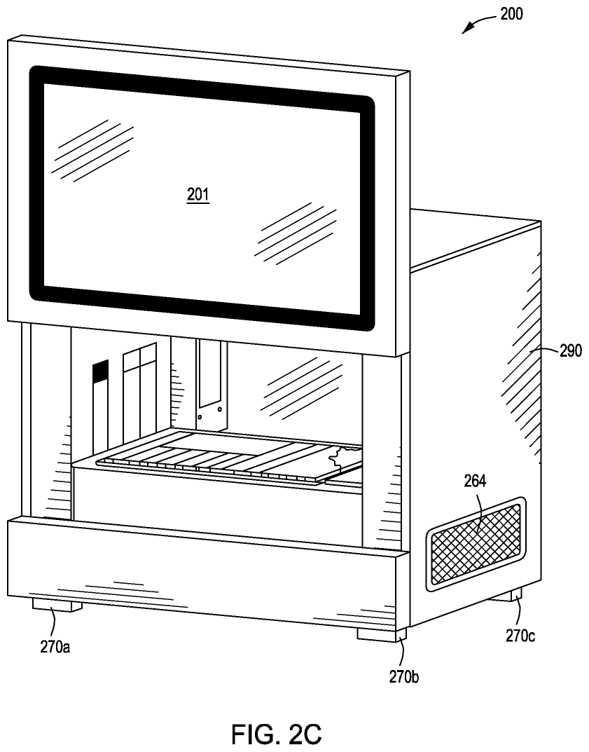

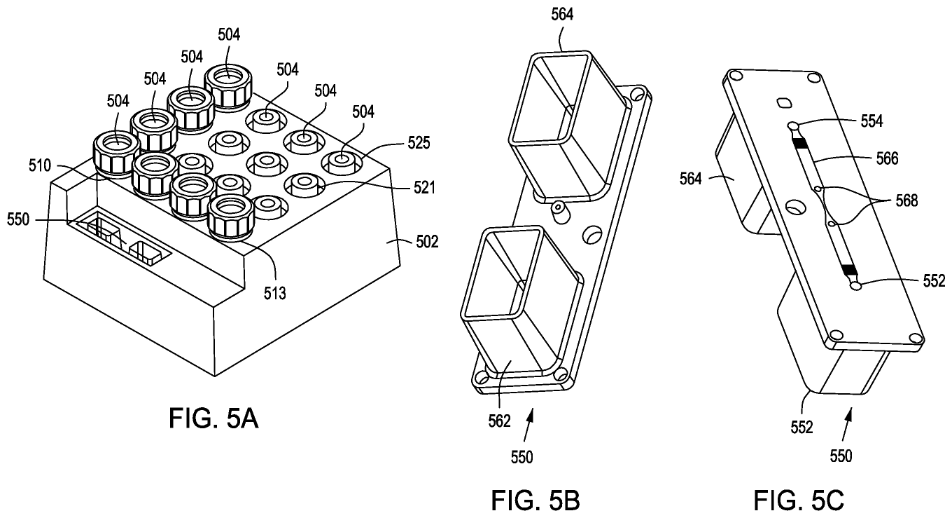

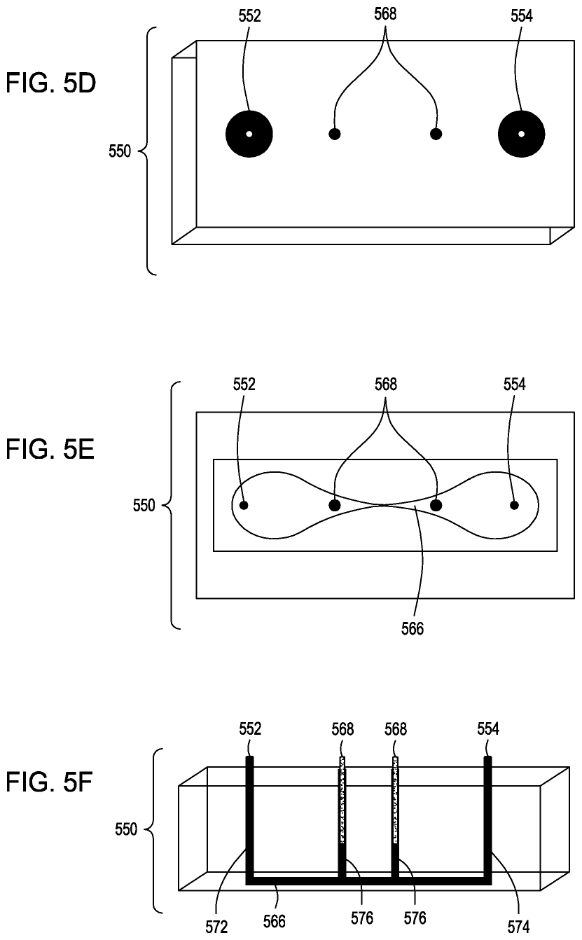

FIG. 2A depicts an exemplary automated multi-module cell processing instrument 200 to, e.g., perform one of the exemplary novel methods using the editing compositions described herein. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV (see, e.g., WO2018015544A1), or Beckman Coulter, Inc. of Fort Collins, CO. (see, e.g., US20160018427A1). Pipette tips may be provided in a pipette transfer tip supply (not shown) for use with the air displacement pipettor 232.

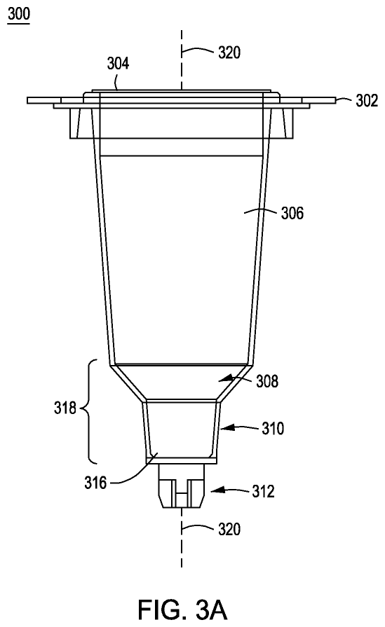

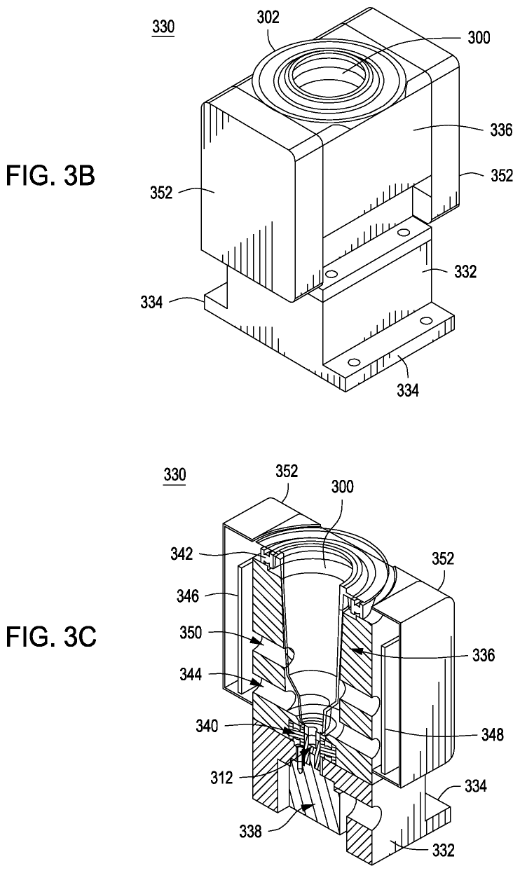

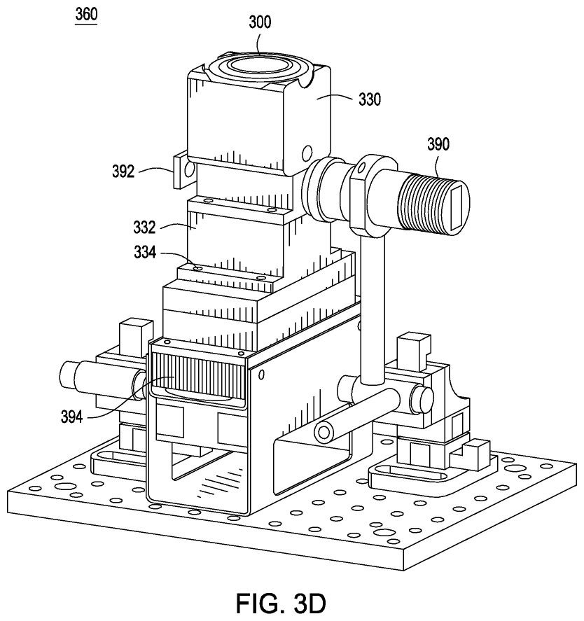

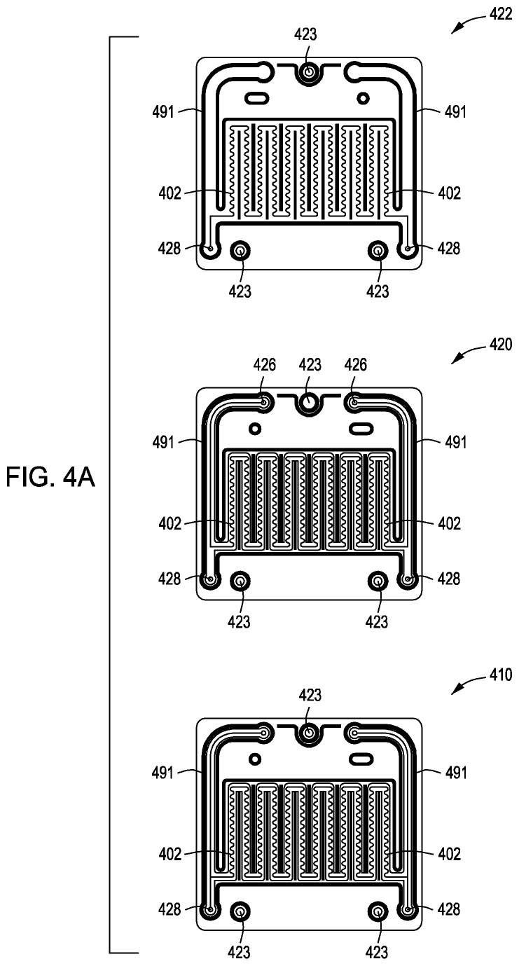

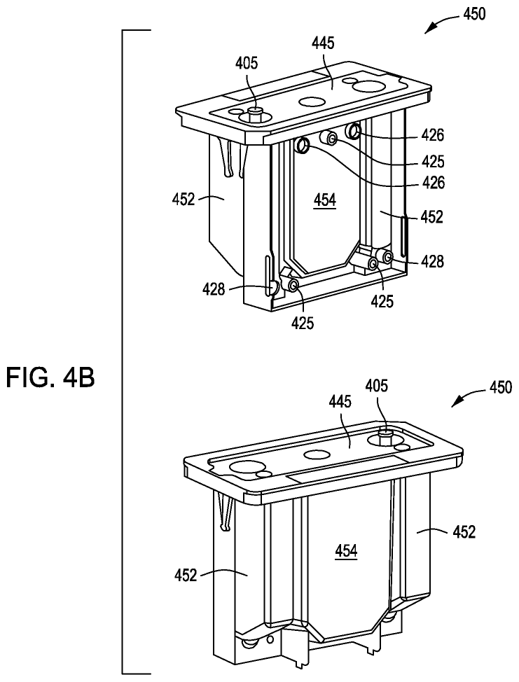

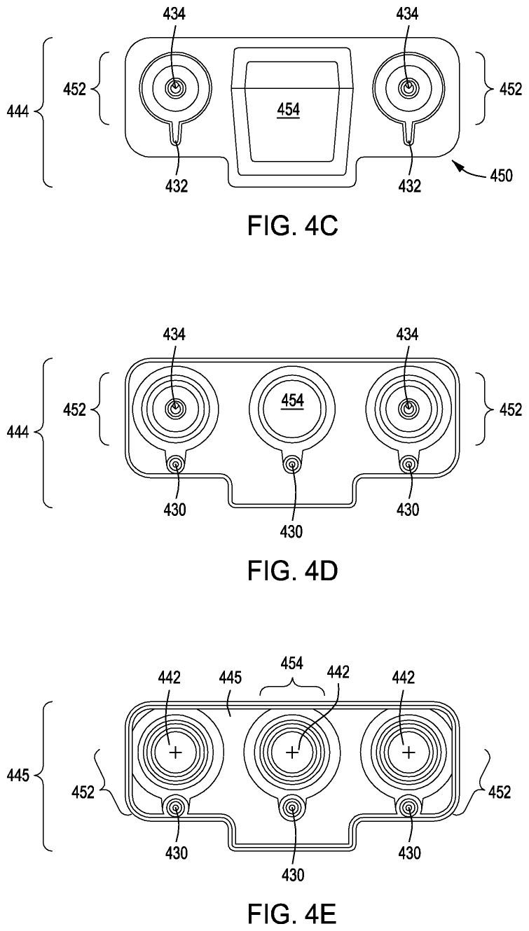

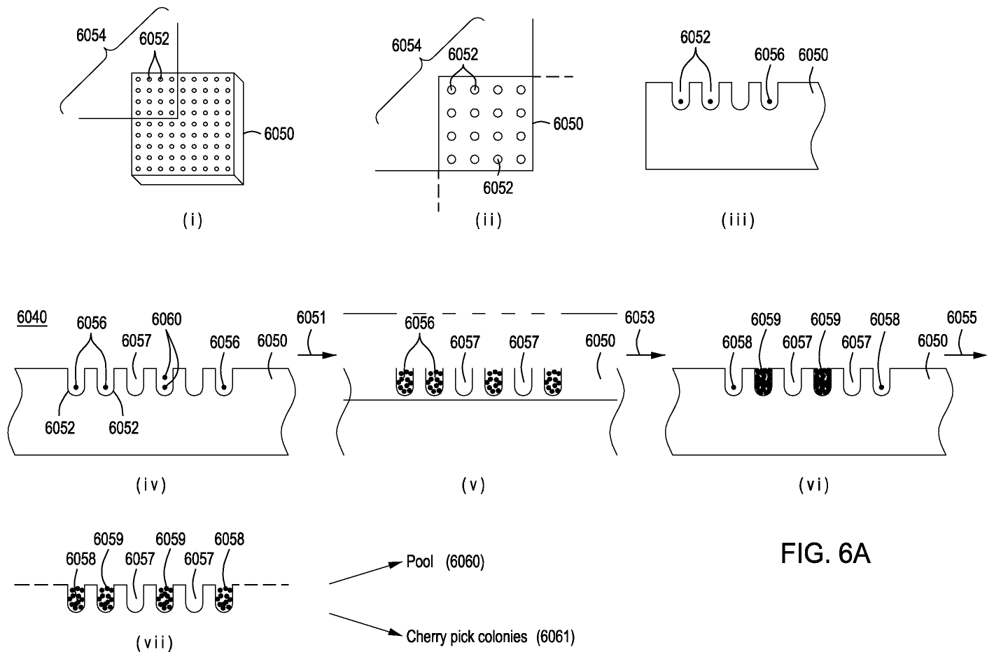

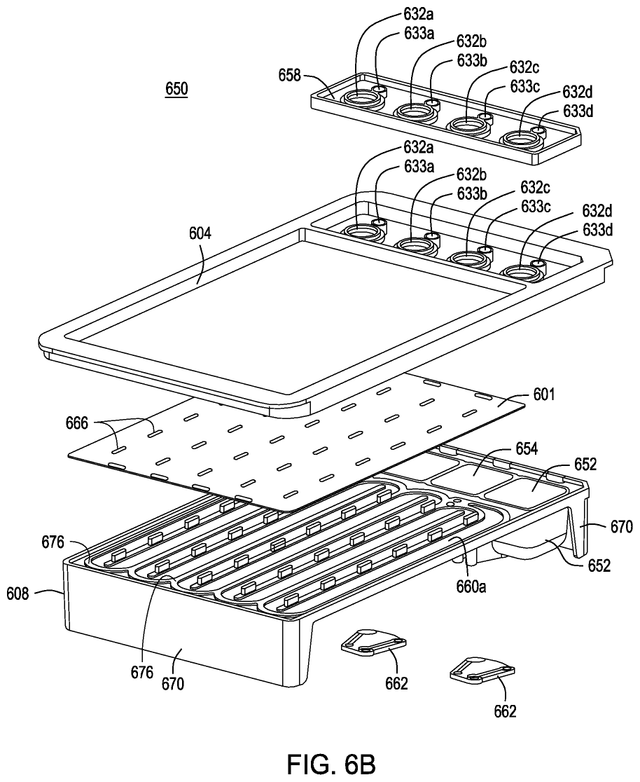

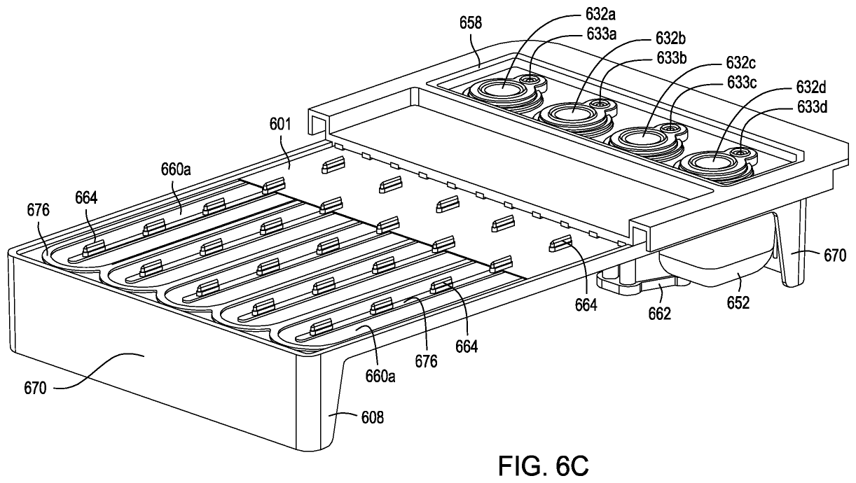

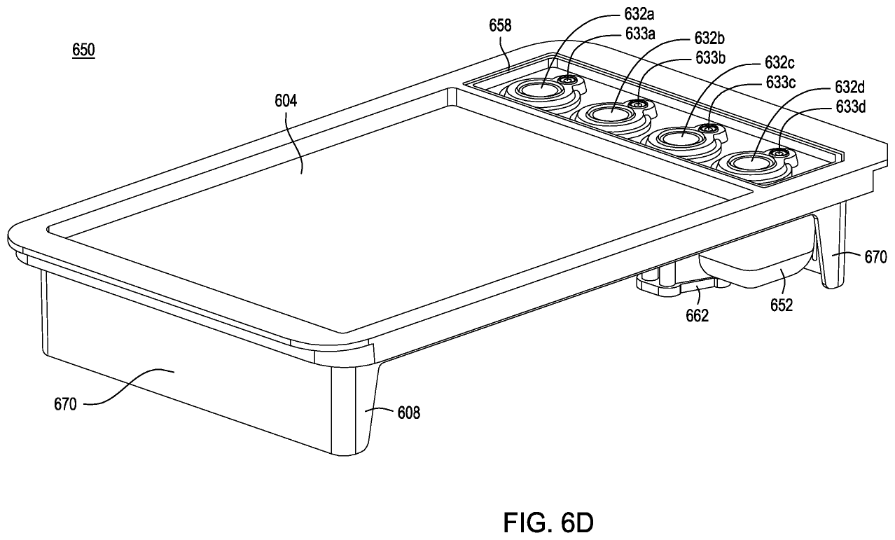

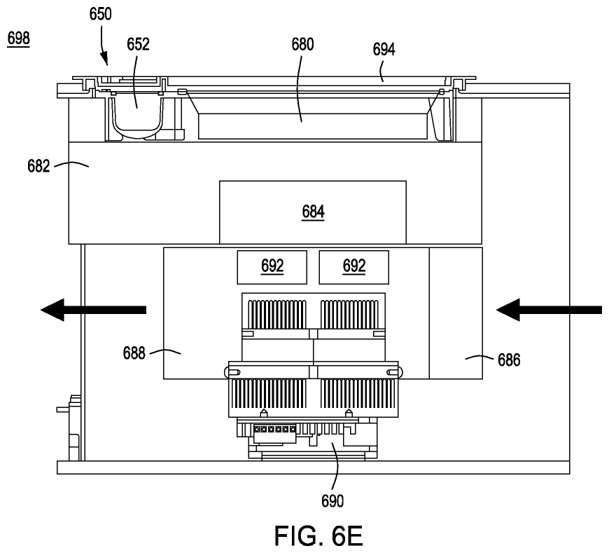

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6F, served by, e.g., robotic liquid handing system 258 and air displacement pipettor 232. Additionally seen is a selection module 220. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge 241, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. No. 10,253,316; 10,329,559; 10,323,242; 10,421,959; 10,465,185; 10,519,437; 10,584,333; 10,584,334; 10,647,982; 10,689,645; 10,738,301; 10,738,663 and U.S. Ser. Nos. 16/412,175 and 16/988,694, all of which are herein incorporated by reference in their entirety.

### The Rotating Cell Growth Module

FIG. 3A shows one embodiment of a rotating growth vial 300 for use with the cell growth device and in the automated multi-module cell processing instruments described herein. The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 400. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330 may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 330 may include additional sensors for measuring, e.g., dissolved oxygen, carbon dioxide, pH, conductivity, and the like. For additional details regarding rotating growth vials and cell growth devices see U.S. Pat. No. 10,435,662, issued 8 Oct. 2019; U.S. Pat. No. 10,443,031, issued 15 Oct. 2019; and U.S. Ser. No. 16/552,981, filed 27 Aug. 2019 and Ser. No. 16/780,640, filed 3 Feb. 2020.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in cell processing systems to perform the methods described herein is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

On the left of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 402 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition, gasket 445 is seen covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretherketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIGS. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again, at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall work flow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 406, collecting the cell culture through a second retentate port 404 into a second retentate reservoir, optionally adding additional or different medium to the cell culture (e.g., subminimal media for inducing a growth-arrested state) and optionally bubbling air or gas through the cell culture, then optionally repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports 404, and the medium/buffer that has passed through the membrane is collected through one or both of the permeate/filtrate ports 406. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells—except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM- (extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeate ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 404 and into the retentate reservoir (not shown). To initiate another “pass”, the cell sample is passed again through the TFF device, this time in a flow direction that is reversed from the first pass. The cell sample is collected by passing through the retentate port 404 and into retentate reservoir (not shown) on the opposite end of the device/module from the retentate port 404 that was used to collect cells during the first pass. Likewise, the medium/buffer that passes through the membrane on the second pass is collected through the permeate port 406 on the opposite end of the device/module from the permeate port 406 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously.

After a final pass in either the growth and concentration processes, fresh medium may be added to the cell sample in the retentate reservoir. The medium used may be a nutrient-rich medium, which may maintain the cells in a growth state, or a subminimal medium, which will induce the cells to shift into a growth-arrested state, as described elsewhere herein. For example, such “final medium” may include M9-type subminimal medium (e.g., M9 medium+/−desired ingredients) or Davis-type Broth (e.g., Davis Minimal Broth+/−desired ingredients). After maintaining the cells in a subminimal medium for a desired amount of time, the cell sample may be transferred to a cell transformation module.

For further information and alternative embodiments on TFFs see, e.g., U.S. Ser. Nos. 62/728,365, filed 7 Sep. 2018; 62/857,599, filed 5 Jun. 2019; and 62/867,415, filed 27 Jun. 2019.

### The Cell Transformation Module

FIG. 5A depicts an exemplary combination reagent cartridge and electroporation device 500 (“cartridge”) that may be used in an automated multi-module cell processing instrument along with the TFF module. In addition, in certain embodiments the material used to fabricate the cartridge is thermally-conductive, as in certain embodiments the cartridge 500 contacts a thermal device (not shown), such as a Peltier device or thermoelectric cooler, that heats or cools reagents in the reagent reservoirs or reservoirs 504. Reagent reservoirs or reservoirs 504 may be reservoirs into which individual tubes of reagents are inserted as shown in FIG. 5A, or the reagent reservoirs may hold the reagents without inserted tubes. Additionally, the reservoirs in a reagent cartridge may be configured for any combination of tubes, co-joined tubes, and direct-fill of reagents.

In one embodiment, the reagent reservoirs or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all reservoirs may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on work flow; that is, the reagents will vary depending on the processes to which the cells are subjected in the automated multi-module cell processing instrument, e.g., protein production, cell transformation and culture, cell editing, etc.

Reagents such as cell samples, enzymes, buffers, nucleic acid vectors, expression cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc. may be positioned in the reagent cartridge at a known position. In some embodiments of cartridge 500, the cartridge comprises a script (not shown) readable by a processor (not shown) for dispensing the reagents. Also, the cartridge 500 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module, if present. The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer.

As described in relation to FIGS. 5B and 5C below, the exemplary reagent cartridges for use in the automated multi-module cell processing instruments may include one or more electroporation devices, preferably flow-through electroporation (FTEP) devices. In yet other embodiments, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif) makes the GENE PULSER XCELL™ line of products to electroporate cells in cuvettes. Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, NV), Beckman Coulter (Fort Collins, CO), etc.

FIGS. 5B and 5C are top perspective and bottom perspective views, respectively, of an exemplary FTEP device 550 that may be part of (e.g., a component in) reagent cartridge 500 in FIG. 5A or may be a stand-alone module; that is, not a part of a reagent cartridge or other module. FIG. 5B depicts an FTEP device 550. The FTEP device 550 has wells that define cell sample inlets 552 and cell sample outlets 554. FIG. 5C is a bottom perspective view of the FTEP device 550 of FIG. 5B. An inlet well 552 and an outlet well 554 can be seen in this view. Also seen in FIG. 5C are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. No. 10,435,713, issued 8 Oct. 2019; U.S. Pat. No. 10,443,074, issued 15 Oct. 2019; U.S. Pat. No. 10,323,258, issued 18 Jun. 2019; U.S. Pat. No. 10,508,288, issued 17 Dec. 2019; U.S. Pat. No. 10,415,058, issued 17 Sep. 2019; and U.S. Ser. Nos. 16/550,790, filed 26 Aug. 2019; and Ser. No. 16/571,080, filed 14 Sep. 2019. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; U.S. Pat. No. 10,406,525, issued 10 Sep. 2019; U.S. Pat. No. 10,478,822, issued 19 Nov. 2019; U.S. Pat. No. 10,576,474, issued 3 Feb. 2020; and U.S. Ser. No. 16/749,757, filed 22 Jan. 2020.

Additional details of the FTEP devices are illustrated in FIGS. 5D-5F. Note that in the FTEP devices in FIGS. 5D-5F the electrodes are placed such that a first electrode is placed between an inlet and a narrowed region of the flow channel, and the second electrode is placed between the narrowed region of the flow channel and an outlet. FIG. 5D shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device. FIG. 5E shows a cutaway view from the top of the FTEP device 550, with the inlet 552, outlet 554, and electrodes 568 positioned with respect to a flow channel 566. FIG. 5F shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretherketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretherketone (PEEK), polysulfone and polyurethane, co-polymers of these and other polymers. Preferred materials include crystal styrene, cyclo-olefin polymer (COP) and cyclic dolphin co-polymers (COC), which allow the device to be formed entirely by injection molding in one piece with the exception of the electrodes and, e.g., a bottom sealing film if present. The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 508 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired-as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the disclosure, the flow-through electroporation device comprises at least one additional electrode which applies a ground potential to the FTEP device.

### Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053. In certain embodiments, the cells may be loaded into the microwells in a subminimal media for inducing a growth-arrested state in the cells during editing, such as M9-type subminimal medium (e.g., M9 medium+/−desired ingredients) or Davis-type Broth (e.g., Davis Minimal Broth+/−desired ingredients).

After transformation and/or editing 6053, many cells in the colonies of cells that have been edited die as a result of the nicks caused by active editing or by fitness effects from the edits themselves and there is a lag in growth for the edited cells that do survive but must repair and recover following editing (microwells 6058), where cells that do not undergo editing thrive (microwells 6059) (vi). All cells are allowed to recover and/or continue grow to establish colonies and normalize, where the colonies of edited cells in microwells 6058 catch up in size and/or cell number with the cells in microwells 6059 that do not undergo editing (vii). Once the cell colonies are normalized, either pooling 6060 of all cells in the microwells can take place, in which case the cells are enriched for edited cells by eliminating the bias from non-editing cells and fitness effects from editing; alternatively, colony size is monitored after editing, and slow growing colonies (e.g., the cells in microwells 6058) may be identified and selected 6061 (e.g., “cherry picked”) resulting in even greater enrichment of edited cells.

In recovering and/or growing the cells, the medium used will depend, of course, on the type of cells being edited—e.g., bacterial, yeast or mammalian- and/or the type of growth state desired for the cells. For example, medium for yeast cell growth includes LB, SOC, TPD, YPG, YPAD, MEM and DMEM. Further, the medium used may be a nutrient-rich medium, which may shift or maintain cells in a growth state, or a subminimal medium for shifting or maintaining cells in a growth-arrested state, such as M9-type subminimal medium (e.g., M9 medium+/−desired ingredients) or Davis-type Broth (e.g., Davis Minimal Broth+/−desired ingredients).

A module useful for performing the method depicted in FIG. 6A is a solid wall isolation, incubation, and normalization (SWIIN) module. FIG. 6B depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6B), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B).

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, CA)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, MA) (also see Levin-Reisman, et al., Nature Methods, 7:737-39 (2010)). Cell growth for, e.g., mammalian cells may be monitored by, e.g., the growth monitor sold by IncuCyte (Ann Arbor, MI) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, NJ); Molecular Devices (QPix 400™ system, San Jose, CA); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the embodiment of the SWIIN module in FIGS. 6B-6D further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6E, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Ser. Nos. 16/399,988, filed 30 Apr. 2019; Ser. No. 16/454,865, filed 26 Jun. 2019; and Ser. No. 16/540,606, filed 14 Aug. 2019. For alternative isolation, incubation and normalization modules, see U.S. Ser. No. 16/536,049, filed 8 Aug. 2019.

### Use of the Automated Multi-Module Cell Processing Instrument

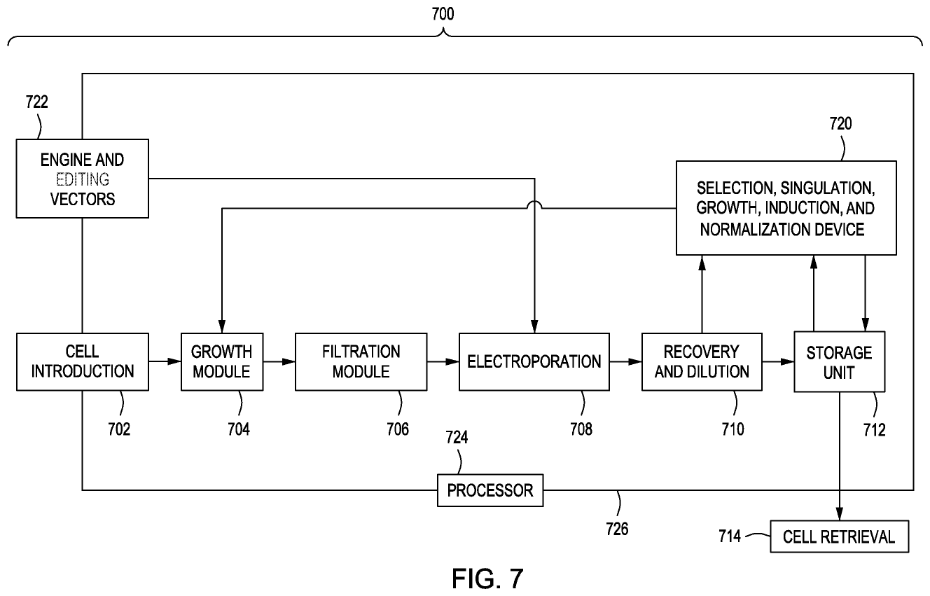

FIG. 7 illustrates an embodiment of a multi-module cell processing instrument. This embodiment depicts an exemplary system that performs recursive and trackable nucleic acid-guided nuclease editing on a cell population. The cell processing instrument 700 may include a housing 726, a reservoir for storing cells to be transformed or transfected 702, and a cell growth module (comprising, e.g., a rotating growth vial) 704. The cells to be transformed are transferred from a reservoir 702 to the cell growth module 704 to be cultured until the cells hit a target OD. Once the cells hit the target OD, the growth module may cool or freeze the cells for later processing or transfer the cells to a cell concentration (e.g., filtration) module 706 where the cells are subjected to buffer exchange and rendered electrocompetent and the volume of the cells may be reduced substantially. Once the cells have been concentrated to an appropriate volume, the cells are transferred to electroporation device 708 or other transformation module. In addition to the reservoir for storing cells 702, the multi-module cell processing instrument includes a reservoir for storing the engine and editing vectors or engine+editing vectors or vectors and proteins to be introduced into the electrocompetent cell population 722. The vector backbones and editing cassettes are transferred to the electroporation device 708, which already contains the cell culture grown to a target OD. In the electroporation device 708, the nucleic acids (or nucleic acids and proteins) are electroporated into the cells. Following electroporation, the cells are transferred into an optional recovery and dilution module 710, where the cells recover briefly post-transformation.

After recovery, the cells may be transferred to a storage module 712, where the cells can be stored at, e.g., 4° C. or −20° C. for later processing, or the cells may be diluted and transferred to a selection/singulation/growth/induction/editing/normalization (SWIIN) module 720. In the SWIIN 720, the cells are arrayed such that there is an average of one to twenty or fifty or so cells per microwell. The arrayed cells may be in selection medium to select for cells that have been transformed or transfected with the editing vector(s). Once singulated, the cells grow through 2-50 doublings and establish colonies. Once colonies are established, editing is induced by providing conditions (e.g., temperature, addition of an inducing or repressing chemical) to induce editing. Editing is then initiated and allowed to proceed, the cells are allowed to grow to terminal size (e.g., normalization of the colonies) in the microwells and then are treated to conditions that cure the editing vector from this round. Once cured, the cells can be flushed out of the microwells and pooled, then transferred to the storage (or recovery) unit 712 or can be transferred back to the growth module 704 for another round of editing. In between pooling and transfer to a growth module, there typically is one or more additional steps, such as cell recovery, medium exchange (rendering the cells electrocompetent), cell concentration (typically concurrently with medium exchange by, e.g., filtration.

Note that the selection/singulation/growth/induction/editing/normalization and curing modules may be the same module, where all processes are performed in, e.g., a solid wall device, or selection and/or dilution may take place in a separate vessel before the cells are transferred to the solid wall singulation/growth/induction/editing/normalization/editing module (SWIIN). Similarly, the cells may be pooled after normalization, transferred to a separate vessel, and cured in the separate vessel. Once the putatively-edited cells are pooled, they may be subjected to another round of editing, beginning with growth, cell concentration and treatment to render electrocompetent, and transformation by yet another donor nucleic acid in another editing cassette via the electroporation module 708.

In electroporation device 708, the cells selected from the first round of editing are transformed by a second set of editing vectors and the cycle is repeated until the cells have been transformed and edited by a desired number of, e.g., CF editing cassettes. The multi-module cell processing instrument exemplified in FIG. 7 is controlled by a processor 724 configured to operate the instrument based on user input or is controlled by one or more scripts including at least one script associated with the reagent cartridge. The processor 724 may control the timing, duration, and temperature of various processes, the dispensing of reagents, and other operations of the various modules of the instrument 700. For example, a script or the processor may control the dispensing of cells, reagents, vectors, and editing oligonucleotides; which editing oligonucleotides are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 7, then the resulting edited culture may go through another (or several or many) rounds of additional editing (e.g., recursive editing) with different editing cassettes (or ribozyme-containing editing cassettes). For example, the cells from round 1 of editing may be diluted and an aliquot of the edited cells edited by editing cassette A may be combined with editing cassette B, an aliquot of the edited cells edited by editing cassette A may be combined with editing cassette C, an aliquot of the edited cells edited by editing cassette A may be combined with editing cassette D, and so on for a second round of editing. After round two, an aliquot of each of the double-edited cells may be subjected to a third round of editing, where, e.g., aliquots of each of the AB-, AC-, AD-edited cells are combined with additional editing cassettes, such as editing cassettes X, Y, and Z. That is, double-edited cells AB may be combined with and edited by editing cassettes X, Y, and Z to produce triple-edited edited cells ABX, ABY, and ABZ; double-edited cells AC may be combined with and edited by editing cassettes X, Y, and Z to produce triple-edited cells ACX, ACY, and ACZ; and double-edited cells AD may be combined with and edited by editing cassettes X, Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the editing vectors comprising the CF editing cassette. “Curing” is a process in which one or more editing vectors used in the prior round of editing is eliminated from the transformed cells. (See, e.g., curing can be accomplished by, e.g., cleaving the editing vector(s) using a curing plasmid thereby rendering the editing vectors nonfunctional; diluting the editing vector(s) in the cell population via cell growth (that is, the more growth cycles the cells go through, the fewer daughter cells will retain the editing vector(s)), or by, e.g., utilizing a heat-sensitive origin of replication on the editing vector. The conditions for curing will depend on the mechanism used for curing; that is, in this example, how the curing plasmid cleaves the editing vector.

### EXAMPLES

The following examples are put forth so as to provide those of ordinary skill in the art with a complete disclosure and description of how to make and use the present disclosure, and are not intended to limit the scope of what the inventors regard as their disclosure, nor are they intended to represent or imply that the experiments below are all of or the only experiments performed. It will be appreciated by persons skilled in the art that numerous variations and/or modifications may be made to the disclosure as shown in the specific aspects without departing from the spirit or scope of the disclosure as broadly described. The present aspects are, therefore, to be considered in all respects as illustrative and not restrictive.

### Example I: Shifting Cells into a Growth-Arrested State During Editing

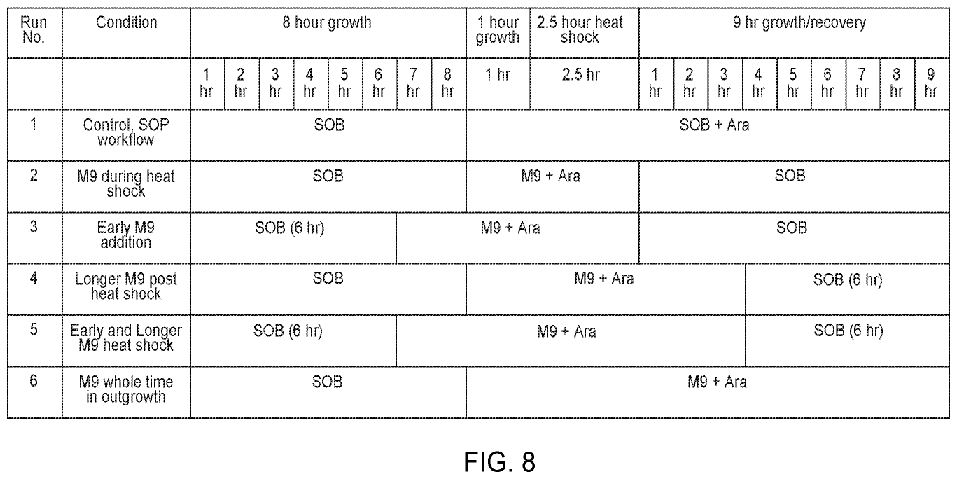

Nucleic acid-guided nuclease editing, utilizing a gRNA and donor template, was carried out in bacterial cells that were induced into a growth-arrested state to demonstrate the effects of growth arrest on editing. Using automated modules as described herein, individual cell populations were exposed to slightly varying growth arrest-inducing conditions upon transformation with editing components, and the editing outcomes thereafter analyzed. The conditions for six editing runs are illustrated in FIG. 8 (shown as “Runs 1-6”, wherein Run 1 is a control).

Briefly, cryopreserved E. coli MG1655 cells previously transformed with an “engine” plasmid were thawed and prepared for transformation with “editing” plasmids. The engine plasmids comprised a MAD7 nuclease under the transcriptional control of a temperature-inducible promoter and a Lambda red recombineering system under the transcriptional control of a pBAD promoter. The editing plasmids comprised a custom strain assessment editing cassette library, wherein each editing cassette was under the transcriptional control of the same temperature-inducible promoter as the nuclease. After transformation with editing components, transformed cell isolates were selected and grown on Super Optimal Broth (SOB) medium for time periods of either 6 hours or 8 hours at 30° C., as shown in FIG. 8.

At either the 6-hour mark or the 8-hour mark, cells of 5/6 experimental runs (Nos. 2-6) were transferred to M9 subminimal broth (Teknova, Hollister, CA, USA) supplemented with 1% arabinose to shift the cells into a growth-arrested state. The cells were maintained in the minimal medium at 30° C. for either 1 hour or 3 hours, after which the cells were exposed to a 2.5-hour 45 C heat shock in the subminimal medium to induce editing. Contrastingly, cells in the control run (Run 1) were transferred to SOB medium supplemented with arabinose and grown for 1 hour before being exposed to the 2.5-hour heat shock to induce editing.

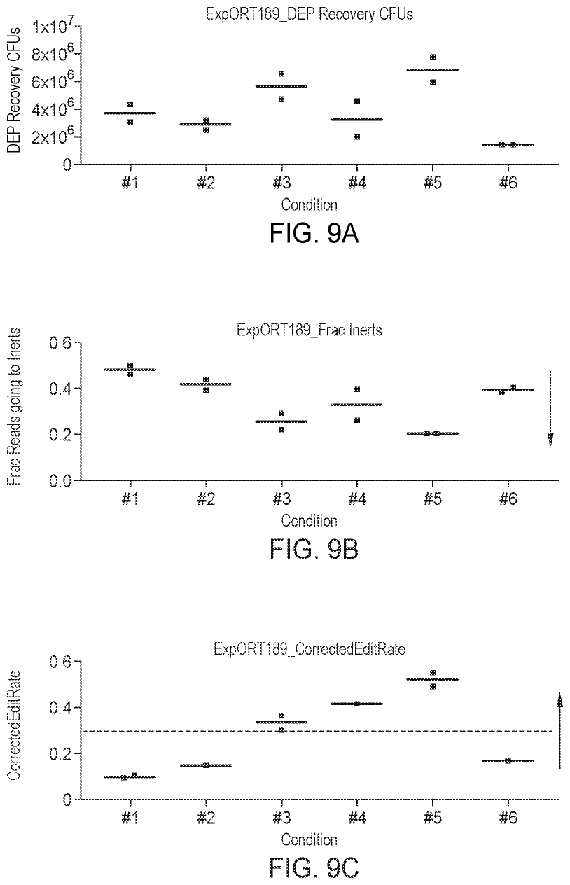

After editing, the cells were allowed to recover and/or grow for 9 hours in either SOB medium, subminimal M9 medium, or subminimal M9 medium followed by transfer to SOB medium at the 3-hour mark. Genomic DNA was purified from the cells using a Promega gDNA extraction kit (Madison, WI, US), and PCR was performed to amplify regions containing the target sites of the editing cassette library. The PCR amplicons were prepared for next-generation sequencing (NGS) using an Illumina TruSeq DNA Sample Preparation Kit according to the manufacturer's instructions, and the samples were sequenced using an Illumina MiSeq using the 2×150 Reagent Kit (Illumina, San Diego, CA, USA) to determine editing rates under the various conditions. The results are depicted in FIGS. 9A-9C.

FIGS. 9A-9C illustrate the number of recovered colony-forming units (CFUs), the fraction of inert cells (i.e., non-edits), and the edit rate, respectively, for each of the six editing runs depicted in FIG. 8. As shown in FIG. 9A, apart from Run 6, growth-arrest conditions resulted in a substantially similar or improved number of recovered CFUs as compared to Run 1, thereby indicating that growth-arrested cells maintained similar or better viability after editing. More importantly, growth-arrest conditions resulted in a reduced fraction of reads going to inerts and an improved editing rate as compared to standard growth conditions, shown in FIGS. 9B and 9C, respectively. These results indicate that shifting cells to a growth-arrested state prior to editing facilitates improved editing outcomes, and particularly, increased editing rates.

Further, the results in FIGS. 9A-9C indicate that earlier onset of growth arrest prior to editing may result in better performance as compared to a later onset thereof. For example, Runs 3 and 5, wherein the cells were transferred from SOB medium to M9 medium at the 6-hour post-transformation mark, showed improved performance as compared to Runs 4 and 6, respectively. It is hypothesized that earlier onset of growth arrest may allow more room for edited cells to grow after editing and better normalization of the cell population.

Still further, the results also indicate that longer maintenance of cells in the growth-arrested state may lead to better editing performance. For example, Run 5, wherein the cells were incubated in M9 medium for 7.5 hours total (before, during, and after editing), showed improved performance as compared to Run 4, wherein the cells were incubated in M9 medium for 5.5 hours total. It is hypothesized that longer maintenance in a growth-arrested state may provide more cells time to incorporate edits, thus giving “inert” cells less of a fitness advantage during post-editing outgrowth.

Yet, shifting the cells back to standard growth conditions for a certain period of time after growth arrest may be necessary in order to arrive at such improved editing outcomes, as evidenced by Run 6. There, the cells were maintained in growth arrest conditions for the duration of the run post-editing, which resulted in diminished editing outcomes as compared to Runs 4 and 5. In light of this, it is postulated that edited cells need nutrient-rich conditions to grow and re-normalize with non-edited cells.

While this disclosure is satisfied by embodiments in many different forms, as described in detail in connection with preferred embodiments of the disclosure, it is understood that the present disclosure is to be considered as exemplary of the principles of the disclosure and is not intended to limit the disclosure to the specific embodiments illustrated and described herein. Numerous variations may be made by persons skilled in the art without departure from the spirit of the disclosure. The scope of the disclosure will be measured by the appended claims and their equivalents. The abstract and the title are snot to be construed as limiting the scope of the present disclosure, as their purpose is to enable the appropriate authorities, as well as the general public, to quickly determine the general nature of the disclosure. In the claims that follow, unless the term “means” is used, none of the features or elements recited therein should be construed as means-plus-function limitations pursuant to 35 U.S.C. § 112, ¶6.

1. A method for performing nucleic acid-guided nuclease editing in a genome of a live cell, the method comprising:
providing an editing system to a cell with a target locus, the editing system comprising:
(a) (i) a nucleic acid-guided nuclease or (ii) a vector encoding the nucleic acid-guided nuclease;
(b) (i) a guide RNA (gRNA) recognizing the target locus or (ii) a nucleic acid encoding the gRNA; and
(c) a donor template comprising a desired edit to be incorporated into the target locus;
inducing the cell into a growth-arrested state; and
providing conditions to allow the editing system to incorporate the desired edit into the target locus.

2. The method of claim 1, wherein the cell is capable of responding to external signals, activating transcription, and producing proteins in the growth-arrested state.

3. The method of claim 1, wherein the conditions to allow the editing system to incorporate the desired edit into the target locus are provided while the cell is in the growth-arrested state.

4. The method of claim 1, wherein the growth-arrested state of the cell is induced via exposure of the cell to one or more stress conditions.

5. The method of claim 4, wherein the one or more stress conditions comprise a chemical stressor.

6. The method of claim 4, wherein the one or more stress conditions comprise a physical stressor.

7. The method of claim 4, wherein the one or more stress conditions comprise a pH change, a temperature change, a high concentration of an organic acid, osmotic stress, oxidative stress, or a nutrient limitation.

8. (canceled)

9. (canceled)

10. (canceled)

11. (canceled)

12. (canceled)

13. The method of claim 7, wherein the nutrient limitation is performed by transferring the cell to a minimal or subminimal media.

14. The method of claim 1, the method further comprising:
providing conditions to allow the cell to recover and grow after allowing the editing system to incorporate the desired edit into the target locus.

15. The method of claim 14, wherein the conditions to allow the cell to recover and grow comprise transferring the cell to nutrient-rich media.

16. A method for performing nucleic acid-guided nuclease editing in a genome of a live cell, the method comprising:
providing a cell with a target locus;
transforming the cell with an editing system, the editing system comprising:
(a) (i) a nucleic acid-guided nuclease or (ii) a vector encoding the nucleic acid-guided nuclease;
(b) (i) a gRNA recognizing the target locus or (ii) a nucleic acid encoding the gRNA; and
(c) a donor template comprising a desired edit to be incorporated into the target locus;
inducing the cell into a growth-arrested state;
providing conditions to allow the editing system to incorporate the desired edit into the target locus; and
inducing the cell into a growth state.

17. The method of claim 16, wherein the cell is capable of responding to external signals, activating transcription, and producing proteins in the growth-arrested state.

18. The method of claim 16, wherein the conditions to allow the editing system to incorporate the desired edit into the target locus are provided while the cell is in the growth-arrested state.

19. The method of claim 16, wherein the growth-arrested state of the cell is induced via exposure of the cell to one or more stress conditions.

20. The method of claim 19, wherein the one or more stress conditions comprise a chemical stressor.

21. The method of claim 19, wherein the one or more stress conditions comprise a physical stressor.

22. The method of claim 19, wherein the one or more stress conditions comprise a pH change, a temperature change, a high concentration of an organic acid, osmotic stress, oxidative stress, or a nutrient limitation.

23. (canceled)

24. (canceled)

25. (canceled)

26. (canceled)

27. (canceled)

28. The method of claim 22, wherein the nutrient limitation is performed by transferring the cell to a subminimal media.

29. The method of claim 16, wherein the conditions to induce the cell into a growth state comprise transferring the cell to nutrient-rich media.

30. The method of claim 1, wherein the cell is a bacterial cell.
